SEQUENCE LISTING (5 sequences)

SEQ ID NO: 1 - protein, 116 aa, Arabidopsis thaliana
MFLPTDYCCRLSDQEYMELVFENGQILAKGQRSNVSLHNQRTKSIMDLYEAEYNEDFMKSIIHGGGGAITNLGDTQVVPQSHVAAAHETNMLESNKHVDGSGSGSGSGSENLYFQG

SEQ ID NO: 2 - protein, 686 aa, Arabidopsis thaliana
MVSGVGGSGGGRGGGRGGEEEPSSSHTPNNRRGGEQAQSSGTKSLRPRSNTESMSKAIQQYTVDARLHAVFEQSGESGKSFDYSQSLKTTTYGSSVPEQQITAYLSRIQRGGYIQPFGCMIAVDESSFRIIGYSENAREMLGIMPQSVPTLEKPEILAMGTDVRSLFTSSSSILLERAFVAREITLLNPVWIHSKNTGKPFYAILHRIDVGVVIDLEPARTEDPALSIAGAVQSQKLAVRAISQLQALPGGDIKLLCDTVVESVRDLTGYDRVMVYKFHEDEHGEVVAESKRDDLEPYIGLHYPATDIPQASRFLFKQNRVRMIVDCNATPVLVVQDDRLTQSMCLVGSTLRAPHGCHSQYMANMGSIASLAMAVIINGNEDDGSNVASGRSSMRLWGLVVCHHTSSRCIPFPLRYACEFLMQAFGLQLNMELQLALQMSEKRVLRTQTLLCDMLLRDSPAGIVTQSPSIMDLVKCDGAAFLYHGKYYPLGVAPSEVQIKDVVEWLLANHADSTGLSTDSLGDAGYPGAAALGDAVCGMAVAYITKRDFLFWFRSHTAKEIKWGGAKHHPEDKDDGQRMHPRSSFQAFLEVVKSRSQPWETAEMDAIHSLQLILRDSFKESEAAMNSKVVDGVVQPCRDMAGEQGIDELGAGTLEKLVDGAGSWSHPQFEKENLYFQGLEHHHHHH

SEQ ID NO: 3 - protein, 676 aa, Arabidopsis thaliana
MVSGVGGSGGGRGGGRGGEEEPSSSHTPNNRRGGEQAQSSGTKSLRPRSNTESMSKAIQQYTVDARLHAVFEQSGESGKSFDYSQSLKTTTYGSSVPEQQITAYLSRIQRGGYIQPFGCMIAVDESSFRIIGYSENAREMLGIMPQSVPTLEKPEILAMGTDVRSLFTSSSSILLERAFVAREITLLNPVWIHSKNTGKPFYAILHRIDVGVVIDLEPARTEDPALSIAGAVQSQKLAVRAISQLQALPGGDIKLLCDTVVESVRDLTGYDRVMVYKFHEDEHGEVVAESKRDDLEPYIGLHYPATDIPQASRFLFKQNRVRMIVDCNATPVLVVQDDRLTQSMCLVGSTLRAPHGCHSQYMANMGSIASLAMAVIINGNEDDGSNVASGRSSMRLWGLVVCHHTSSRCIPFPLRYACEFLMQAFGLQLNMELQLALQMSEKRVLRTQTLLCDMLLRDSPAGIVTQSPSIMDLVKCDGAAFLYHGKYYPLGVAPSEVQIKDVVEWLLANHADSTGLSTDSLGDAGYPGAAALGDAVCGMAVAYITKRDFLFWFRSHTAKEIKWGGAKHHPEDKDDGQRMHPRSSFQAFLEVVKSRSQPWETAEMDAIHSLQLILRDSFKESEAAMNSKVVDGVVQPCRDMAGEQGIDELGAGSGSGLNDIFEAQKIEWHEHHHHHH

SEQ ID NO: 4 - protein, 380 aa, synthetic construct
  REGION 1..380: note = H57 Heavy Chain - PIF
MEFGLSWVFLVALFRGVQCEVYLVESGGDLVQPGSSLKVSCAASGFTFSDFWMYWVRQAPGKGLEWVGRIKNIPNNYATEYADSVRGRFTISRDDSRNSIYLQMNRLRVDDTAIYYCTRAGRFDHFDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTGAGSGSGSGSGSMMFLPTDYCCRLSDQEYMELVFENGQILAKGQRSNVSLHNQRTKSIMDLYEAEYNEDFMKSIIHGGGGAITNLGDTQVVPQSHVAAAHETNMLESNKHVDGSGSGSGSGSENLYFQGHHHHHH

SEQ ID NO: 5 - protein, 238 aa, synthetic construct
  REGION 1..238: note = H57 Light Chain
MKYLLPTAAAGLLLLAAQPAMAYELIQPSSASVTVGETVKITCSGDQLPKNFAYWFQQKSDKNILLLIYMDNKRPSGIPERFSGSTSGTTATLTISGAQPEDEAAYYCLSSYGDNNDLVFGSGTQLTVLRGRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2025/0249278 A1
(43) Pub. Date: Aug. 7, 2025

(54) METHODS FOR CONTROLLING THE TUMOR CELL KILLING BY LIGHT

(71) Applicants: INSTITUT NATIONAL DE LA SANTÉ ET DE LA RECHERCHE MÉDICALE, Paris (FR); CENTRE NATIONAL DE LA RECHERCHE SCIENTIFIQUE, Paris (FR); UNIVERSITÉ D'AIX MARSEILLE, Marseille (FR)

(72) Inventors: Rémi LASSERRE, Marseille Cedex 09 (FR); Morgane JAEGER, Marseille Cedex 09 (FR)

(21) Appl. No.: 18/856,303

(22) PCT Filed: Apr. 13, 2023
(86) PCT No.: PCT/EP2023/059716

§ 371 Date: Oct. 11, 2024

(30) Foreign Application Priority Data
Apr. 14, 2022 (EP) 22305545.0

(51) Int. Cl. A61N 5/06; A61P 35/00; C07K 14/415; C07K 16/28; C07K 16/30

(52) U.S. Cl. CPC A61N 5/062; A61P 35/00; C07K 14/415; C07K 16/2809; C07K 16/30; C07K 2317/31; C07K 2317/55; C07K 2317/569; C07K 2317/75; C07K 2319/00

(57) ABSTRACT

The inventors have developed a new system of optogenetics-based recombinant system allowing to target tumor cells to control in space and time tumor cell lysis by cytotoxic T lymphocytes (CTLs) with light. To do so, they have coupled tumor-specific antigen antibody to a photoreceptor protein that can bind an optogenetic domain linked to a Fab fragment derived from an agonistic antibody targeting the TCR. They demonstrated that these new system allow the spatio-temporal control of the tumor cell killing by CTLs in vitro, in response to light. The present invention relates to methods of activating on demand an immune cell or a plurality of immune cells, and methods for treating cancer.

### FIELD OF THE INVENTION

The present invention relates to methods and systems for controlling the tumor cell killing by light. The present invention relates thus to method for treating cancer.

### BACKGROUND OF THE INVENTION

Oncological phototherapy, including current photodynamic therapy (PDT), developmental photoactivated chemotherapy (PACT) and photothermal therapy (PTT), shows promising photo-efficacy for superficial and internal tumours [1]. Photodynamic therapy (PDT), for example, was discovered more than 100 years ago, and has since become a well-studied therapy for a wide range of medical conditions, such as malignant cancers including head and neck, lung, bladder and particular skin [2]. Photodynamic therapy uses a drug that is activated by light, called a photosensitizer or photosensitizing agent, to kill cancer cells. The light can come from a laser or other source, such as LEDs. Photodynamic therapy is most often used as a local treatment, which means it treats a specific part of the body. Indeed, the dual application of light and photochemotherapeutic agents allows accurate cancer targeting and low invasiveness. Damage to normal cells is limited but photodynamic therapy can still cause burns, swelling, pain, and scarring in the treatment area. For now, phototherapy was essentially used for chemotherapy. Studying the influence of the dynamics of signals perceived by immune cells on the quality of their response is becoming a new field of investigation in all areas of biomedical research such as immunotherapy.

Because of their pivotal role in immunity, T cells have been a central target for the development of immunotherapies, particularly in the field of cancer research. New therapies targeting T cell activation against tumor cells have been continuously developed in recent years (Waldman, A. D et al. Nat Rev Immunol, 2020) These therapies are based on different strategies, including engineered patient-derived T cells with chimeric antigenic receptors (CARs), T cell activation checkpoint inhibitory antibodies, or bispecific T cell engagers (BiTEs) bridging cytotoxic T cells to tumor cells and promoting tumor cell killing (Blanco, B. et al. Clin Cancer Res. 2021). They showed a remarkable efficiency on different types of cancer and have radically changed the prognosis of patients. However, these molecules often induce serious side effects as they may unleash the T cell compartment not only at the tumor site, but also in healthy tissue or at the systemic level (Das, S et al. J Immunother Cancer. 2019). These secondary effects, including life-threatening syndromes like cytokine storm, healthy tissue destruction or autoimmunity are called immune-related adverse events (irAEs). They are becoming a central challenge in immuno-oncology because they are frequent, can be irreversible, sometimes fatal and very difficult to predict (Wang, D. Y et al. JAMA Oncol. 2018). Therefore, there is a great need to develop molecules whose activity can be precisely controlled in time and space to limit IrAEs.

Some recent study illustrates that T cells respond to minute-scale oscillations of activation signal by stimulating an optogenetically controlled chimeric antigen receptor (optoCAR) (O'Donoghue et al. PNAS, 2021). This elegant study clearly showed that CAR T cells integrate pulsatile stimulations, the dynamics of which influences the magnitude of the T cell response. However, these studies that directly question the influence of the dynamics of minute-scaled stimulations on the T cell activation are based on CAR and not on normal TCR stimulations. In addition, transducing such receptors in T cells requires T cell pre-activation and retroviral infection. Therefore, methods to provide an accurate and reversible spatiotemporal control of TCR stimulation in untouched primary T cells are still missing.

Thus, the development of new tool allowing precise control of tumor cell lysis in time and space is necessary. The present invention extend phototherapies to agonistic antibodies and bispecific immune cell engagers.

### SUMMARY OF THE INVENTION

The present invention relates to a light-controlled molecular system comprising:
a. at least one recombinant protein comprising a variable domain of an antibody that is fused at its c-terminal end to a compound that can interact with a photoactivable agent in a light-dependent manner, and
b. at least one molecule specific for an tumor-antigen that is fused at its c-terminal end to the photoactivable agent.

The present invention also related to a method for treating tumor in a subject in need thereof, comprising administering to said subject (a) at least one recombinant protein comprising a variable domain of an antibody that is fused at its c-terminal end to a compound that can interact with a photoactivable agent in a light-dependent manner, and (b) at least one said tumor-antigen antibody that is fused at its c-terminal end to the photoactivable agent; and exposing the tumor with a suitable wavelength of light wherein said exposition allows the formation of a complex binding the recombinant protein to the tumor-antigen antibody.

In particular, the present invention is defined by the claims.

### DETAILED DESCRIPTION OF THE INVENTION

The inventors have previously invented the Light-inducible T cell engager (LiTe or OptoFab), a new class of optogenetics-based recombinant molecules able to reversibly trigger TCR signaling in response to specific wavelength of light as described in the patent WO2020/070288. These molecules are composed of a Fab fragment derived from an agonistic antibody targeting the TCR, linked to an optogenetic domain that allows its light induced oligomerization/immobilization. They have demonstrated that the OptoFab system provides a highly potent light-controllable T cell activation system whose reversibility permits the precise scaling in time of the TCR stimulations.

The inventors has now developed a new version of the OptoFab system allowing to target tumor cells to control in space and time tumor cell lysis by cytotoxic T lymphocytes (CTLs) with light. To do so, they have coupled tumor-specific antigen antibody to the photoreceptor protein of the optogenetic domain linked to the Fab fragment derived from an agonistic antibody targeting the TCR. They demonstrated that this new system allow the spatio-temporal control of the tumor cell killing by CTLs in vitro, in response to light.

### System of the Invention

Accordingly, the first object of the present invention relates to a light-controlled molecular system comprising:
a. at least one recombinant protein comprising a variable domain of an antibody that is fused at its c-terminal end to a compound that can interact with a photoactivable agent in a light-dependent manner, and
b. at least one molecule specific for an tumor-antigen that is fused at its c-terminal end to the photoactivable agent.

In other words, the present invention relates to a kit comprising:
a. at least one recombinant protein comprising a variable domain of an antibody that is fused at its c-terminal end to a compound that can interact with a photoactivable agent in a light-dependent manner, and
b. at least one molecule specific for an tumor-antigen that is fused at its c-terminal end to the photoactivable agent.

As used herein, the term “variable domain” refers to both variable domains of immunoglobulin light chains and variable domains of heavy chain of an antibody.

As used herein the term “antibody” or “immunoglobulin” refers to immunoglobulin molecules and immunologically active portions of immunoglobulin molecules, i.e., molecules that contain an antigen binding site that immunospecifically binds an antigen. As such, the term antibody encompasses not only whole antibody molecules, but also antibody fragments as well as variants (including derivatives) of antibodies and antibody fragments. The term also encompasses antibodies that are naturally devoid light chain that can be found e.g. in Camelid mammals. Thus the term encompasses single domain antibodies. The term also encompasses Fab, F(ab′)2, Fab′, dsFv, diabodies and scFv. The term also encompasses antibody mimetic such as designed ankyrin repeat protein (DARPin).

In natural antibodies, two heavy chains are linked to each other by disulfide bonds and each heavy chain is linked to a light chain by a disulfide bond. There are two types of light chain, lambda (l) and kappa (κ). There are five main heavy chain classes (or isotypes) which determine the functional activity of an antibody molecule: IgM, IgD, IgG, IgA and IgE. Each chain contains distinct sequence domains. The light chain includes two domains, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from nonhypervariable or framework regions (FR) can participate to the antibody binding site or influence the overall domain structure and hence the combining site. Complementarity Determining Regions or CDRs refer to amino acid sequences which together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, typically includes six CDRs, comprising the CDR set from each of a heavy and a light chain V region. Framework Regions (FRs) refer to amino acid sequences interposed between CDRs.

As used herein, the term “single domain antibody” has its general meaning in the art and refers to the single heavy chain variable domain of antibodies of the type that can be found in Camelid mammals which are naturally devoid of light chains. Such single domain antibody are also called VHH or “Nanobody®”. For a general description of single domain antibodies, reference is made to EP 0 368 684, Ward et al. (Nature 1989 Oct. 12; 341 (6242): 544-6), Holt et al., Trends Biotechnol., 2003, 21 (11): 484-490; and WO 06/030220, WO 06/003388. The amino acid sequence and structure of a single domain antibody can be considered to be comprised of four framework regions or “FRs” which are referred to in the art and herein as “Framework region 1” or “FR1”; as “Framework region 2” or “FR2”; as “Framework region 3” or “FR3”; and as “Framework region 4” or “FR4” respectively; which framework regions are interrupted by three complementary determining regions or “CDRs”, which are referred to in the art as “Complementarity Determining Region for “CDR1”; as “Complementarity Determining Region 2” or “CDR2” and as “Complementarity Determining Region 3” or “CDR3”, respectively. Accordingly, the single domain antibody can be defined as an amino acid sequence with the general structure: FR1-CDR1-FR2-CDR2-FR3-CDR3-FR4 in which FR1 to FR4 refer to framework regions 1 to 4 respectively, and in which CDR1 to CDR3 refer to the complementarity determining regions 1 to 3.

As used herein, the term “F(ab′)2” refers to an antibody fragment having a molecular weight of about 100,000 and antigen binding activity, which is slightly larger than the Fab bound via a disulfide bond of the hinge region, among fragments obtained by treating IgG with a protease, pepsin.

As used herein, the term “Fab′” refers to an antibody fragment having a molecular weight of about 50,000 and antigen binding activity, which is obtained by cutting a disulfide bond of the hinge region of the F(ab′)2.

As used herein, the term “single chain Fv” (“scFv”) polypeptide is a covalently linked VH:VL heterodimer which is usually expressed from a gene fusion including VH and VL encoding genes linked by a peptide-encoding linker.

As used herein, the term “dsFv” is a VH:VL heterodimer stabilised by a disulfide bond. Divalent and multivalent antibody fragments can form either spontaneously by association of monovalent scFvs, or can be generated by coupling monovalent scFvs by a peptide linker, such as divalent sc (Fv)2.

As used herein, the term “diabodies” refers to small antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites.

Thus, in some embodiments, the variable domain comprising in the recombinant protein of the invention is selected from the group consisting of VH domains, VL domains, or single domain antibodies (sdAbs).

In some embodiments, the variable domain comprising in the recombinant protein of the invention is a single domain antibody.

In some embodiments, the variable domain comprising in the recombinant protein of the invention is a VH domain of a monoclonal antibody.

As used herein, the term “monoclonal antibody” refer to a preparation of antibody molecules of single molecular composition. A monoclonal antibody composition displays a single binding specificity and affinity for a particular epitope.

In some embodiments, the recombinant protein of the present invention comprises a Fab fragment wherein the VH domain of the Fab fragment is fused at its c-terminal end to a factor that can interact with a photoreceptor protein in a light-dependent manner.

As used herein, the term “Fab fragment” has its general meaning in the art and refers to a monovalent fragment of an antibody consisting of the VL, VH, CL and CH1 domains. Fab fragments can be typically obtained, e.g., by treating an IgG antibody with papain. It is indeed well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). An antibody from which the Fc region has been enzymatically cleaved, or which has been produced without the Fc region, designated a Fab fragment, retains one of the antigen binding sites of an intact antibody molecule. Thus, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

In some embodiment, the Fab fragment comprising in the recombinant protein derives from an antibody able to inhibit the function of its specific receptor.

In some embodiment, the Fab fragment derives from an antibody specific for a receptor and whose the monovalent form is not able to block the receptor function.

In some embodiments, the Fab fragment comprising in the recombinant protein derives from an agonistic antibody.

In some embodiments, the Fab fragment comprising in the recombinant protein derives from an agonistic antibody whose the monovalent form is not able to induce the biological signaling activity of the receptor.

In some embodiments, the variable domain comprising in the recombinant protein of the invention is an agonistic single domain antibody.

In some embodiments, the variable domain comprising in the recombinant protein of the invention is an agonistic single domain antibody whose the monovalent form is not able to induce the biological signaling activity of the receptor.

As used herein, the term ‘agonistic antibody’ describes an antibody that is an agonist i.e. that is capable of stimulating the biological signalling activity of a receptor. As used herein, the term “receptor” has its general meaning in the art and denotes a cell-associated protein that binds to a bioactive molecule (i.e., a ligand) and mediates the effect of the ligand on the cell. Membrane-bound receptors are characterized by a multi-domain structure comprising an extracellular ligand-binding domain and an intracellular effector domain that is typically involved in signal transduction.

In some embodiments, the agonistic antibody is specific for a receptor of an immune cell. In particular, the antibody may be specific for an immune cell regulatory molecule such as CD3, CD4, CD8, CD25, CD28, CD26, CTLA-4, ICOS, or CD11a. Other suitable antigens include but are not limited to those associated with immune cells including T cell-associated molecules, such as TCR/CD3 or CD2; NK cell-associated targets such as NKG2D, FcγRIIIa (CD16), CD38, CD44, CD56, or CD69; granulocyte-associated targets such as FcγRI (CD64), FcγRI (CD89), and CR3 (CD11b/CD18); monocyte/macrophage-associated targets (such as FcγRI (CD64), FcγRI (CD89), CD3 (CD11b/CD18), or mannose receptor; dendritic cell-associated targets such as FcγRI (CD64) or mannose receptor; and erythrocyte-associated targets such as CRI (CD35).

In some embodiments, the agonistic antibody is specific for a TCR. As used herein, the term “TCR” has its general meaning in the art and refers to the molecule found on the surface of T cells that is responsible for recognizing antigens bound to MHC molecules. During antigen processing, antigens are degraded inside cells and then carried to the cell surface in the form of peptides bound to major histocompatability complex (MHC) molecules (human leukocyte antigen HLA molecules in humans). T cells are able to recognize these peptide-MHC complex at the surface of professional antigen presenting cells or target tissue cells such as β cells in TID. There are two different classes of MHC molecules: MHC Class I and MHC Class II that deliver peptides from different cellular compartments to the cell surface that are recognized by CD8+ and CD4+ T cells, respectively. The T cell receptor or TCR is the molecule found on the surface of T cells that is responsible for recognizing antigens bound to MHC molecules. The TCR heterodimer consists of an alpha and beta chain in 95% of T cells, whereas 5% of T cells have TCRs consisting of gamma and delta chains. Engagement of the TCR with antigen and MHC results in activation of its T lymphocyte through a series of biochemical events mediated by associated enzymes, co-receptors, and specialized accessory molecules. Each chain of the TCR is a member of the immunoglobulin superfamily and possesses one N-terminal immunoglobulin (Ig)-variable (V) domain, one Ig-constant (C) domain, a transmembrane region, and a short cytoplasmic tail at the C-terminal end. The constant domain of the TCR consists of short connecting sequences in which a cysteine residue forms a disulfide bond, making a link between the two chains. The structure allows the TCR to associate with other molecules like CD3 which possess three distinct chains (γ, δ, and ε) in mammals and the ζ-chain. These accessory molecules have negatively charged transmembrane regions and are vital to propagating the signal from the TCR into the cell. The CD3 chains, together with the TCR, form what is known as the TCR complex. The signal from the TCR complex is enhanced by simultaneous binding of the MHC molecules by a specific co-receptor. On helper T cells, this co-receptor is CD4 (specific for class II MHC); whereas on cytotoxic T cells, this co-receptor is CD8 (specific for class I MHC). The co-receptor not only ensures the specificity of the TCR for an antigen, but also allows prolonged engagement between the antigen presenting cell and the T cell and recruits essential molecules (e.g., LCK) inside the cell involved in the signaling of the activated T lymphocyte. The term “T-cell receptor” is thus used in the conventional sense to mean a molecule capable of recognising a peptide when presented by an MHC molecule. The molecule may be a heterodimer of two chains α and β (or optionally γ and δ) or it may be a recombinant single chain TCR construct. The variable domain of both the TCR α-chain and β-chain have three hypervariable or complementarity determining regions (CDRs). CDR3 is the main CDR responsible for recognizing processed antigen. Its hypervariability is determined by recombination events that bring together segments from different gene loci carrying several possible alleles. The genes involved are V and J for the TCR α-chain and V, D and J for the TCR β-chain. Further amplifying the diversity of this CDR3 domain, random nucleotide deletions and additions during recombination take place at the junction of V-J for TCR α-chain, thus giving rise to V(N)J sequences; and V-D and D-J for TCR β-chain, thus giving rise to V(N)D(N)J sequences. Thus, the number of possible CDR3 sequences generated is immense and accounts for the wide capability of the whole TCR repertoire to recognize a number of disparate antigens. At the same time, this CDR3 sequence constitutes a specific molecular fingerprint for its corresponding T cell. The CDR3 amino acid and nucleotide sequences of the TCR characterized by the inventors are listed in the following Table A. Rearranged nucleotide sequences are presented as V segments (underlined) followed by (ND) N segments (not underlined; N additions denoted in bold) and then by J segments (underlined), as annotated using the IMGT database (www.imgt.org).

In some embodiments, the agonistic antibody is specific for a costimulatory receptor. As used herein, the term “costimulatory receptor” includes receptors which transmit a costimulatory signal to an immune cell. In some embodiments, costimulatory receptor is selected from the group consisting of CD134 (OX40), CD137 (4-1BB), CD28, GITR, CD27, CD70, ICOS, RANKL, TNFRSF25 (DR3), CD258 (LIGHT), CD40, HVEM, and the like.

In some embodiments, the agonistic antibody is specific for a receptor selected from the group consisting of CD1a, CD1b, CD1c, CD1d, CD1e, CD2, CD3delta, CD3epsilon, CD3gamma, CD4, CD5, CD6, CD7, CD8alpha, CD8beta, CD9, CD10, CD11a, CD11b, CD11c, CDw12, CD13, CD14, CD15u, CD16a, CD16b, CDw17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42a, CD42b, CD42c, CD42d, CD43, CD44, CD44R, CD45, CD46, CD47R, CD48, CD49a, CD49b, CD49c, CD49d, CD49e, CD49f, CD50, CD51, CD52, CD53, CD54, CD55, CD56, CD57, CD58, CD59, CD60a, CD60b, CD60c, CD61, CD62E, CD62L, CD62P, CD63, CD64, CD65, CD65s, CD66a, CD66b, CD66c, CD66d, CD66e, CD66f, CD68, CD69, CD70, CD71, CD72, CD73, CD74, CD75, CD75s, CD77, CD79a, CD79b, CD80, CD81, CD82, CD83, CD84, CD85, CD86, CD87, CD88, CD89, CD90, CD91, CD92, CDw93, CD94, CD95, CD96, CD97, CD98, CD99, CD100, CD101, CD102, CD103, CD104, CD105, CD106, CD107a, CD107b, CD108, CD109, CD110, CD111, CD112, CDw113, CD114, CD115, CD116, CD117, CD118, CDw119, CD120a, CD120b, CD121a, CDw121b, CD122, CD123, CD124, CDw125, CD126, CD127, CDw128a, CDw128b, CD129, CD130, CD131, CD132, CD133, CD134, CD135, CDw136, CDw137, CD138, CD139, CD140a, CD140b, CD141, CD142, CD143, CD144, CDw145, CD146, CD147, CD148, CDw149, CD150, CD151, CD152, CD153, CD154, CD155, CD156a, CD156b, CDw156C, CD157, CD158, CD159a, CD159c, CD160, CD161, CD162, CD162R, CD163, CD164, CD165, CD166, CD167a, CD168, CD169, CD170, CD171, CD172a, CD172b, CD172g, CD173, CD174, CD175, CD175s, CD176, CD177, CD178, CD179a, CD179b, CD180, CD181, CD182, CD183, CD184, CD185, CDw186, CD191, CD192, CD193, CD195, CD196, CD197, CDw198, CDw199, CDw197, CD200, CD201, CD202b, CD203c, CD204, CD205, CD206, CD207, CD208, CD209, CDw210, CD212, CD213a1, CD213a2, CDw217, CDw218a, CDw218b, CD220, CD221, CD222, CD223, CD224, CD225, CD226, CD227, CD228, CD229, CD230, CD231, CD232, CD233, CD234, CD235a, CD235b, CD235ab, CD236, CD236R, CD238, CD239, CD240CE, CD240D, CD240DCE, CD241, CD242, CD243, CD244, CD245, CD246, CD247, CD248, CD249, CD252, CD253, CD254, CD256, CD257, CD258, CD261, CD262, CD263, CD264, CD265, CD266, CD267, CD268, CD269, CD271, CD272, CD273, CD274, CD275, CD276, CD277, CD278, CD279, CD280, CD281, CD282, CD283, CD284, CD289, CD292, CDw293, CD294, CD295, CD296, CD297, CD298, CD299, CD300a, CD300c, CD300e, CD301, CD302, CD303, CD304, CD305, CD306, CD307, CD309, CD312, CD314, CD315, CD316, CD317, CD318, CD319, CD320, CD321, CD322, CD324, CDw325, CD326, CDw327, CDw328, CDw329, CD331, CD332, CD333, CD334, CD335, CD336, CD337, CDw338 and CD339.

In some embodiment, the agonistic antibody is specific for a TCR, and in particular for a human TCR.

In some embodiment, the agonistic antibody is a TCR Beta monoclonal antibody, and in particular a human TCR Beta single domain antibody.

In some embodiment, the agonistic antibody is a TCR Beta monoclonal antibody who react with alpha and beta TCR but not with gamma and delta TCR.

In some embodiment, the agonistic antibody is monoclonal antibody H57-597, as described in Kubo, R. T., et al. (1989). J. Immunol. 142 (8): 2736-2742.

In some embodiment, the agonistic antibody is specific for CD3 and in particular for human CD3.

In some embodiment, the agonistic antibody is specific for CD3epsilon and in particular for human CD3epsilon

In some embodiment, the agonistic antibody is monoclonal antibody 145-2C11, as described in Leo O et al. (1986). Proc. Nat. Acad. Sci USA. Vol 84. pp 1374-1378.

In some embodiment, the agonistic single domain antibody is specific for a TCR, and in particular for human TCR.

In some embodiment, the agonistic single domain antibody is a TCR Beta single domain antibody, and in particular a human TCR Beta single domain antibody.

In some embodiment, the agonistic single domain antibody is a TCR Beta single domain antibody who react with alpha and beta TCR but not with gamma and delta TCR.

In some embodiment, the agonistic single domain antibody is specific for CD3, and in particular for human CD3.

In some embodiment, the agonistic single domain antibody is specific for CD3epsilon, and in particular for human CD3epsilon

In particular embodiment, the molecule specific for an tumor-antigen is a tumor-antigen targeting antibody.

Thus, in particular embodiment, the present invention relates to an light-controlled molecular system comprising:
a. at least one recombinant protein comprising a variable domain of an antibody that is fused at its c-terminal end to a compound that can interact with a photoactivable agent in a light-dependent manner, and
b. at least one tumor-antigen targeting antibody that is fused at its c-terminal end to the photoactivable agent.

As used herein, the term “tumor-antigen targeting antibody” has its general meaning in the art and refers to antibodies (Ab) or antibodies mimetics, more preferably monoclonal antibodies (mAb), that recognizes and binds to tumor membrane proteins, block cell signaling, and induce tumor-killing through Fc-driven innate immune responses. According to the invention, the tumor-antigen targeting antibody include tumor-associated antigen targeting and tumor-specific antigen targeting. Tumor-associated antigens (TAAs) are relatively restricted to tumor cells. According to the invention, the tumor-antigen targeting antibody mimetics include tumor-associated antigen targeting and tumor-specific antigen targeting.

In particular embodiment, the tumor-antigen targeting antibody is a single domain antibody (sdAb).

Thus, in some embodiments, the present invention relates to a light-controlled molecular system comprising:
a. at least one recombinant protein comprising a variable domain of an antibody that is fused at its c-terminal end to a compound that can interact with a photoactivable agent in a light-dependent manner, and
b. at least one tumor-antigen targeting single domain antibody that is fused at its c-terminal end to the photoactivable agent.

In some embodiments, the present invention relates to a light-controlled molecular system comprising:
a. at least one recombinant protein comprising a variable domain of an agonistic antibody specific for TCR beta or CD3epsilon that is fused at its c-terminal end to a compound that can interact with a photoactivable agent in a light-dependent manner, and
b. at least one tumor-antigen targeting single domain antibody that is fused at its c-terminal end to the photoactivable agent.

In some embodiments, the present invention relates to a light-controlled molecular system comprising:
a. at least one recombinant protein comprising an agonistic single domain antibody that is fused at its c-terminal end to a compound that can interact with a photoactivable agent in a light-dependent manner, wherein the agonistic single domain antibody is specific for TCR beta or CD3epsilon, and
b. at least one tumor-antigen targeting single domain antibody that is fused at its c-terminal end to the photoactivable agent.

In particular embodiment, the molecule specific for an tumor-antigen is an tumor-antigen targeting antibody mimetics, and more particularly an tumor-antigen targeting DARPin.

In other words, in particular embodiment, the molecule specific for an tumor-antigen is a tumor-antigen targeting antibody mimetic.

Thus, in particular embodiment, the present invention relates to an light-controlled molecular system comprising:
a. at least one recombinant protein comprising a variable domain of an antibody that is fused at its c-terminal end to a compound that can interact with a photoactivable agent in a light-dependent manner, and
b. at least one tumor-antigen targeting antibody mimetic that is fused at its c-terminal end to the photoactivable agent.

In some embodiments, the present invention relates to a light-controlled molecular system comprising:
a. at least one recombinant protein comprising a variable domain of an agonistic antibody specific for TCR beta or CD3epsilon that is fused at its c-terminal end to a compound that can interact with a photoactivable agent in a light-dependent manner, and
b. at least one tumor-antigen targeting single domain antibody mimetic that is fused at its c-terminal end to the photoactivable agent.

As used herein, the term “antibody mimetics” refers to compounds that can specifically bind antigens in a manner analogous to that of the antigen-antibody. Antibody mimetics includes but are not limited to affibody molecules, affilins, affimers, affitins, alphabodies, anticalins, avimers, DARPins, fynomers, gastrobodies, kunitz domain peptide, monobodies, nanoCLAMPS, optimers, repebodies pronectin, centyrins and obodies.

In some embodiments, the tumor-antigen targeting antibody mimetics is a DARPin.

As used herein, the term “designed ankyrin repeat proteins” or “DARPin” hast its general meaning in the art and refers to genetically engineered antibody mimetic proteins typically exhibiting highly specific and high-affinity target protein binding. They are derived from natural ankyrin repeat proteins, one of the most common classes of binding proteins in nature, which are responsible for diverse functions such as cell signaling, regulation and structural integrity of the cell.

Tumor-specific antigens (TSAs) are unique to tumor cells. A regularly updated database of those antigenic peptides effectively presented by tumor cells can be found on the http://www.cancerimmunity.org/website [3]

In some embodiments, the tumor-antigen targeting antibody is specific for a human tumor-antigen.

In some embodiments, the tumor-antigen targeting antibody is specific for a tumor-antigen selected from the group consisting of human epithelial cell adhesion molecule (hEpCAM), Isocitrate dehydrogenase [NADP] cytoplasmic (IDH1), Aldehyde Dehydrogenase 1 Family Member A1 (ALDH1), CD274, CD45, cyclin D1 (BCL1), Dickkopf-Related Protein 1 (DKK-1), Enhancer Of Zeste Homolog 2 (EZH2), Heat Shock Protein Family H (Hsp110) Member 1 (HSPH1), Kallikrein Related Peptidase 4 (KLK4), Kinesin Family Member 20A (KIF20A), Papillomavirus Regulatory Factor 1 (PBF), Vascular Endothelial Growth Factor A (VEGF), B Cell Maturation Antigen (BCMA), ICOS, CD19, CD20, CD24, CD27 CD28, CD33, CD37, CD38, CD157, CD40, CD44, CD47, CD86, CD122, CD123, CD137, CD160, Human 5′-nucleotidase (NT5E), Recombinant Human Lysosome-associated membrane glycoprotein 1 (LAMP1), Tumor necrosis factor receptor superfamily member 4 (TNFRSF4), Tumor necrosis factor receptor superfamily member 18 (TNFRSF18) CTL-recognized antigen on melanoma (CAMEL), Differentiation antigen melanoma 6 (DAM-6), Differentiation antigen melanoma 10 (DAM-10), G antigen 1 (GAGE-1), G antigen 2 (GAGE-2), G antigen 3 (GAGE-3), G antigen 4 (GAGE-4), G antigen 5 (GAGE-5), G antigen 6 (GAGE-6), G antigen 7 (GAGE-7), G antigen 8 (GAGE-8), Interleukin 13 receptor alpha2 chain (IL-13Rα2), Melanoma antigen A1 (MAGE-A1), Melanoma antigen A2 (MAGE-A2), Melanoma antigen A3 (MAGE-A3), Melanoma antigen A4 (MAGE-A4), Melanoma antigen A6 (MAGE-A6), Melanoma antigen A9 (MAGE-A9), Melanoma antigen A10 (MAGE-A10), Melanoma antigen A12 (MAGE-A12), Melanoma antigen C1 (MAGE-C1), Melanoma antigen C2 (MAGE-C2), NA cDNA clone of patient M88 (NA88-A), New York esophageous 1 (NY-ESO-1), Synovial sarcoma, X breakpoint 2 (SSX-2), Synovial sarcoma, X breakpoint 4 (SSX4), Taxol resistant associated protein 3 (TRAG-3), carcinoembryonic antigen (CEA), Epithelial cell adhesion molecule (Ep-CAM), Basal cell adhesion molecule (BCAM), orphan G protein-coupled receptor, class C group 5 member D (GPRC5D), Fc Receptor-Like 5 (FCRL5), DLL3 (Delta Like Canonical Notch Ligand 3) Melanoma-Associated ME20 Antigen (GP100), mammaglobin-A, Melanoma antigen recognized by T cells-1/melanoma antigen A (Melan-A/MART-1), Melanocortin 1 receptor (MC1R), Ocular albinism type 1 protein (OA1), Prostate-specific antigen (PSA), Tyrosinase-related protein 1 (TRP-1), Tyrosinase-related protein 2 (TRP-2), tyrosinase, adipophilin, alpha-fetoprotein (AFP), interferon-inducible protein absent in melanoma 2 (AIM-2), acute lymphoblastic leukemia (ALL), 707 alanine proline (707-AP), acute promyelocytic leukemia (APL), adenocarcinoma antigen recognized by T cells 4 (ART-4), B antigen (BAGE), Ephrin type-A receptor 2 (EphA2), Ephrin type-A receptor 3 (EphA3), Fibroblast growth factor 5 (FGF5), Glycoprotein 250 (G250), Alpha-Mannoside Beta-1,6-N-Acetylglucosaminyltransferase V (GnTV), hER2, human signet-ring tumor 2 (HST-2), human telomerase reverse transcriptase (hTERT), M-CSF, mucin-1 (MUC1), mucin-2 (MUC2), mucin-16 (MUC16), mucin-17 (MUC17), Preferentially expressed antigen of melanoma (PRAME), Prostate-specific membrane antigen (PSMA), protein 15 (p15), protein 53 (p53), renal antigen (RAGE), renal ubiquitous 1 (RU1), renal ubiquitous 2 (RU2), squamous antigen rejecting tumor 1 (SART-1), squamous antigen rejecting tumor 2 (SART-2), squamous antigen rejecting tumor 3 (SART-3), SRY-Box Transcription Factor 10 (SOX10), Wilms Tumor 1 (WT1), 707 alanine proline (707-AP), α-actinin-4, β-catenin, Casein Kinase 1 Alpha 1 (CSNKIA1), Cyclin Dependent Kinase Inhibitor 2A (CDKN2A), Caseinolytic Mitochondrial Matrix Peptidase Proteolytic Subunit (CLPP), Colorectal Tumor-Associated Antigen-1 (COA-1), Elongation factor 2 (ELF2), Melanoma Ag recognized by T cells-2 (MART2), Melanoma ubiquitous mutated 1 (MUM1), Melanoma ubiquitous mutated 2 (MUM2), Melanoma ubiquitous mutated 3 (MUM3), myosin, OS9 Endoplasmic Reticulum Lectin (OS-9), Transforming Protein P21 (K-ras), Neuroblastoma RAS Viral Oncogene Homolog (N-ras), O-Linked N-acetylglucosamine transferase gene (OGT), TGFαRII, L antigen (LAGE-1), annexin II, cell division cycle 27 (CDC27), Neo-Poly(A) Polymerase (neo-PAP), Receptor-type protein-tyrosine phosphatase kappa (PTPRK), TGFβRII, Adaptor Related Protein Complex 2 Subunit Sigma 1 (AP2S1), BBX High Mobility Group Box Domain Containing (ARTC1), B-Raf Proto-Oncogene, Serine/Threonine Kinase (B-RAF), caspase-5 (CASP-5), caspase-8 (CASP-8), elongation factor 2, FLT3, fibronectin 1 (FN1), Fibronectin Type III Domain Containing 3B (FNDC3B), Growth Arrest Specific 7 (GAS7), Glycoprotein Nmb (GPNMB), HAUS Augmin Like Complex Subunit 3 (HAUS3), HLA-A11, HLA-A2, Hydroxysteroid Dehydrogenase Like 1 (HSDL1), Heat shock protein 70-2 (HSP70-2), Matrilin-1 (MATN), Malic Enzyme 1 (ME1), Protein Phosphatase 1 Regulatory Subunit 3B (PPPIR3B), Peroxiredoxin 5 (PRDX5), Ubiquitin Protein Ligase E3 Component N-Recognin 4 (RBAF600), Sirtuin 2 (SIRT2), triosephosphate isomerase, Cancer/Testis Antigen 37 (CT37/FMRINB), cylcin-A1, Cancer/Testis Antigen 83 (KK-LC-1), Cancer/Testis Antigen KM-HN-1 (KM-HN-1), LDL Receptor Related Protein Associated Protein 1 (LRPAP1), lymphocyte Antigen 6 Family Member K (LY6K), sarcoma antigen (SAGE), Ankyrin Repeat Domain 30A (NY-BR-1), prostatic Acid Phosphatase (PAP), prostate stem-cell antigen (PSCA), A-kinase anchor protein 4 (AKAP-4) Bcl-2-Like Protein (BCLX), WD Repeat Domain 46 (BING-4), calcitonin (CALCA), Programmed Cell Death 1 Ligand 1 (PDL1), Leukocyte Common Antigen (LCA), Cleavage And Polyadenylation Specific Factor 1 (CPSF), Dickkopf-Related Protein 1, cyclin D1, cyclin-dependent kinase 4 (CDK4), nectin-2, nectin-4, Enhancer Of Zeste Homolog 2 (EZH2), P53-Binding Protein Mdm2 (MDM2), Matrix Metallopeptidase 2 (MMP2), Matrix Metallopeptidase 7 (MMP-7), glypican-3, hepsin, Hepatocyte Cell Adhesion Molecule (HEPACAM), Heat Shock Protein Family H Member 1 (HSPH1), Indoleamine 2,3-Dioxygenase 1 (IDO1), HLA-G, Mitochondrial Ribosomal Protein S4 (IMP3), carboxylesterase 2 (CES2), kallikrein 4, Mitotic Kinesin-Like Protein 2 (KIF20A), lengsin, midkine, Paired Box Protein Pax-5 (PAX5), Cancer/Testis Antigen 92 (PLAC1), Regulator Of G-Protein Signaling 5 (RGS5), Ras Homolog Family Member C (RHOC), Ring Finger Protein 43 (RNF43), Doublecortin Domain Containing 2 (RU2), secernin 1, survivin, telomerase, Six Transmembrane Epithelial Antigen Of The Prostate 1 (STEAP1), and Trophoblast Glycoprotein (TPBG).

In some embodiments, the tumor-antigen targeting antibody is specific for TRP1 (TRP1-targeting antibody).

In some embodiments, the TRP1-targeting antibody is monoclonal antibody TA99, as described in Thomson T M, et al. (1985). J Invest Dermatol. 1985 August; 85 (2): 169-74.

In some embodiments, the tumor-antigen targeting antibody is specific for EpCAM (EpCAM-targeting antibody).

Methods for producing antibodies are well known in the art. For instance, monoclonal antibodies may be generated using the method of Kohler and Milstein (Nature, 256:495, 1975). To prepare monoclonal antibodies useful in the invention, a mouse or other appropriate host animal is immunized at suitable intervals (e.g., twice-weekly, weekly, twice-monthly or monthly) with the appropriate antigenic forms (i.e. receptor of interest). The animal may be administered a final “boost” of antigen within one week of sacrifice. It is often desirable to use an immunologic adjuvant during immunization. Suitable immunologic adjuvants include Freund's complete adjuvant, Freund's incomplete adjuvant, alum, Ribi adjuvant, Hunter's Titermax, saponin adjuvants such as QS21 or Quil A, or CpG-containing immunostimulatory oligonucleotides. Other suitable adjuvants are well-known in the field. The animals may be immunized by subcutaneous, intraperitoneal, intramuscular, intravenous, intranasal or other routes. A given animal may be immunized with multiple forms of the antigen by multiple routes. Briefly, the recombinant receptor of interest may be provided by expression with recombinant cell lines. Recombinant forms of the polypeptides may be provided using any previously described method. Following the immunization regimen, lymphocytes are isolated from the spleen, lymph node or other organ of the animal and fused with a suitable myeloma cell line using an agent such as polyethylene glycol to form a hydridoma. Following fusion, cells are placed in media permissive for growth of hybridomas but not the fusion partners using standard methods. Following culture of the hybridomas, cell supernatants are analyzed for the presence of antibodies of the desired specificity, i.e., that selectively bind the antigen. Suitable analytical techniques include ELISA, flow cytometry, immunoprecipitation, and western blotting. Other screening techniques are well-known in the field. Preferred techniques are those that confirm binding of antibodies to conformationally intact, natively folded antigen, such as non-denaturing ELISA, flow cytometry, and immunoprecipitation.

Many agonistic antibodies are known in the art. For instance, anti-OX40 antibodies are described, for example, in U.S. Pat. Nos. 8,614,295; 7,501,496; and 8,283,450, incorporated herein by reference in their entirety for the disclosure of anti-OX40 antibodies. Anti-4-1BB antibodies are described, for example, in U.S. Pat. Nos. 6,569,997; 6,974,863; and 8,137,667, incorporated herein by reference in their entirety for the disclosure of anti-4-1BB antibodies. Anti-CD28 antibodies are described, for example, in U.S. Pat. Nos. 7,585,960; 8,334,102, and 7,723,482, incorporated herein by reference in their entirety for the disclosure of anti-CD28 antibodies. Anti-GITR antibodies are described, for example, in U.S. Pat. Nos. 7,812,135 and 8,388,967, incorporated herein by reference in their entirety for the disclosure of anti-GITR antibodies. Anti-CD27 antibodies are described, for example, in U.S. Pat. No. 8,481,029, incorporated herein by reference in its entirety for the disclosure of anti-CD28 antibodies. Anti-CD70 antibodies are described, for example, in U.S. Pat. Nos. 8,337,838; 8,124,738; and 7,491,390, incorporated herein by reference in their entirety for the disclosure of anti-CD70 antibodies. Anti-ICOS antibodies are described, for example, in U.S. Pat. Nos. 7,521,532 and 8,318,905, incorporated herein by reference in their entirety for the disclosure of anti-ICOS antibodies. Anti-RANKL antibodies are described, for example, in U.S. Pat. Nos. 7,411,050; 8,414,890, and 8,377,690, incorporated herein by reference in their entirety for the disclosure of anti-RANKL antibodies. An exemplary anti-RANKL antibody is denosumab. Anti-TNFRSF25 (DR3) antibodies are described, for example, in U.S. Patent Publication Nos. US20130330360, and US20120014950 incorporated herein by reference in their entirety for the disclosure of anti-DR3 antibodies. Anti-CD258 (LIGHT) antibodies are described, for example, in U.S. Patent Publication Nos. US20130315913 and US20090214519, incorporated herein by reference in their entirety for the disclosure of anti-LIGHT antibodies. Anti-CD40 antibodies are described, for example, in U.S. Pat. Nos. 8,669,352; 8,637,032; 8,591,900; 8,492,531; 8,388,971; 8,303,955; 7,790,166; 7,666,422; 7,563,442; 7,537,763; and 7,445,780, incorporated herein by reference in their entirety for the disclosure of anti-CD40 antibodies. Anti-HVEM antibodies are described, for example, in U.S. Pat. Nos. 6,573,058, and 8,440,185, incorporated herein by reference in their entirety for the disclosure of anti-HVEM antibodies.

As used herein, the term “photoactivable agent” refers to a compound (proteins or small molecules) that can be triggered by light stimulation.

In particular embodiment, the photoactivable agent is not IRDye 700DX (IR700).

In particular embodiment, the photoactivable agent is not Prussian blue or Prussian blue-derivatives.

In some embodiment, the tumor-antigen targeting antibody is conjugated to the photoactivable agent by any suitable means, as will be apparent to those of skill in the art. In some embodiments, the variable domain and the compound that can interact with a photoactivable agent in a light-dependent manner are fused to each other by any suitable means, as will be apparent to those of skill in the art. In some embodiments, the variable domain and the compound that can interact with a photoactivable agent in a light-dependent manner are fused to each other directly (i.e. without use of a linker) or via a linker. In some embodiments, the tumor-antigen targeting antibody and the photoactivable agent are fused to each other directly or via a linker. The linker is typically a linker peptide and will, according to the invention, be selected so as to allow binding of the polypeptide to the heterologous polypeptide. Suitable linkers will be clear to the skilled person based on the disclosure herein, optionally after some limited degree of routine experimentation. Suitable linkers are described herein and may—for example and without limitation-comprise an amino acid sequence, which amino acid sequence preferably has a length of 2 or more amino acids. Typically, the linker has 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acids. The linker sequence may be a naturally occurring sequence or a non-naturally occurring sequence. One useful group of linker sequences are linkers derived from the hinge region of heavy chain antibodies as described in WO 96/34103 and WO 94/04678. Other examples are poly-alanine linker sequences such as Ala-Ala-Ala. Further preferred examples of linker sequences are Gly/Ser linkers of different length including (gly4ser)3, (gly4ser)4, (gly4ser), (gly3ser), gly3, and (gly3ser2)3.

In some embodiment, the click chemistry can be used to conjugated the tumor-antigen antibody to the photoactivable agent and/or to fused the variable domain and the compound that can interact with a photoactivable agent in a light-dependent manner.

As used herein, the term “click-chemistry” has its general meaning in the art and refers to bioconjugations giving high yield and selectivity biomolecules by carbon-hetero bond formation reactions. Click chemistry include but are not limited to azide-alkyne cycloaddition such as copper(I)-catalyzed azide-alkyne cycloaddition, strain-promoted azide-alkyne cycloaddition; Strain-promoted alkyne-nitrone cycloaddition; reaction of strained alkenes such as allkene and azide [3+2] cycloaddition, alkene and tetrazine inverse-demand Diels-Alder, and Alkene and tetrazole photoclick reaction.

In some embodiments, the tumor-antigen targeting antibody is conjugated with biotin, and fused to the biotinylated photoactivable agent via streptavidin, avidin or neutravidin. It thus contemplated that modified forms of avidin or streptavidin are employed to bind or capture the biotinylated tumor-associated antigen targeting antibody and the biotinylated-photoactivable agent. A number of modified forms of avidin or streptavidin that bind biotin specifically are known. Such modified forms of avidin or streptavidin include, e.g., physically modified forms (Kohanski, R. A. and Lane, M. D. (1990) Methods Enzymol. 194-200), chemically modified forms such as nitro-derivatives (Morag, E., et al., Anal. Biochem. 243 (1996) 257-263) and genetically modified forms of avidin or streptavidin (Sano, T., and Cantor, C. R., Proc. Natl. Acad. Sci. USA 92 (1995) 3180-3184).

In some embodiments, at least two tumor-antigen targeting antibody that is fused at its c-terminal end to the photoactivable agent are administered in the method of the present invention.

In some embodiments, two tumor-antigen targeting antibody are conjugated with biotin, and fused each to one biotinylated photoactivable agent via one molecule selected in the group of streptavidin, avidin or neutravidin.

In some embodiments, two tumor-antigen targeting antibody are conjugated with biotin, and fused each to biotinylated-tumor-antigen via streptavidin, avidin or neutravidin in order to form a complex composed of one streptavidin, avidin or neutravidin, two biotinylated-tumor-antigen targeting antibody and two biotinylated photoactivable agent, as described in FIG. 1A.

In some embodiment, the compound that can interact with a photoactivable agent in a light-dependent manner is a factor that can interact with a photoreceptor protein in a light-dependent manner and the photoactivable agent fused to antigen-associated tumor antibody is the photoreceptor protein.

Thus in particular embodiment, the invention refers to a light-controlled molecular system comprising:
a. at least one recombinant protein comprising a variable domain of an antibody that is fused at its c-terminal end to a factor that can interact with a photoreceptor protein in a light-dependent manner, and
b. at least one tumor-antigen targeting antibody that is fused at its c-terminal end to the photoreceptor protein.

Thus in particular embodiment, the invention refers to a light-controlled molecular system comprising:
a. at least one recombinant protein comprising a variable domain of an antibody specific for TCR beta or CD3epsilon, wherein the single domain antibody is fused at its c-terminal end to a factor that can interact with a photoreceptor protein in a light-dependent manner, and
b. at least one tumor-antigen targeting single domain antibody that is fused at its c-terminal end to the photoreceptor protein.

In some embodiments, factors that interact with a photoreceptor protein in a light-dependent manner are not particularly limited and include any proteins or protein fragments that are capable of binding to a cognate photoreceptor protein in a light-dependent manner, i.e. that bind to a photo-activated from of the photoreceptor, but not to a photo-inactivated from. In some embodiments, said factor is selected from the group consisting of Phytochrome Interacting Factors (PIFs), FHY1/FHL, Phytochrome kinase substrate 1 (PKS1), nucleoside diphosphate kinase 2 (NDPK2), cryptochromes such as CRY1 and CRY2, Aux/IAA proteins, phosphatases such as FyPP and PAPP5, E3 ubiquitin ligases such as COP1, and ARR4. Preferably, said factor is selected from the group consisting of PIF1, PIF2, PIF3, PIF4, PIF5, PIF6, and PIF7, wherein PIF6 is particularly preferred. Even more preferably, said factor consists of the first 100 amino-terminal amino acids of PIF6. In one particular example, said factor can be PIF6 derived from Arabidopsis thaliana. In some embodiments, the factor comprises an amino acid sequence that has at least 90% of identity with the amino acid sequence as set forth in SEQ ID NO: 1. In some embodiment, the factor comprises or refers to an amino acid sequence as set forth in SEQ ID NO:1.
SEQ ID NO: 1
MFLPTDYCCRLSDQEYMELVFENGQILAKGQRSNVSLHNQRTKSIMDLYEAEYNEDFMKSIIHGGGGAI TNLGDTQVVPQSHVAAAHETNMLESNKHVDGSGSGSGSGSENLYFQG

According to the invention a first amino acid sequence having at least 90% of identity with a second amino acid sequence means that the first sequence has 90; 91; 92; 93; 94; 95; 96; 97; 98; 99 or 100% of identity with the second amino acid sequence. Sequence identity is frequently measured in terms of percentage identity (or similarity or homology); the higher the percentage, the more similar are the two sequences. Methods of alignment of sequences for comparison are well known in the art. Various programs and alignment algorithms are described in: Smith and Waterman, Adv. Appl. Math., 2:482, 1981; Needleman and Wunsch, J. Mol. Biol., 48:443, 1970; Pearson and Lipman, Proc. Natl. Acad. Sci. U.S.A., 85:2444, 1988; Higgins and Sharp, Gene, 73:237-244, 1988; Higgins and Sharp, CABIOS, 5:151-153, 1989; Corpet et al. Nuc. Acids Res., 16:10881-10890, 1988; Huang et al., Comp. Appls Biosci., 8:155-165, 1992; and Pearson et al., Meth. Mol. Biol., 24:307-31, 1994). Altschul et al., Nat. Genet., 6:119-129, 1994, presents a detailed consideration of sequence alignment methods and homology calculations. By way of example, the alignment tools ALIGN (Myers and Miller, CABIOS 4:11-17, 1989) or LFASTA (Pearson and Lipman, 1988) may be used to perform sequence comparisons (Internet Program® 1996, W. R. Pearson and the University of Virginia, fasta20u63 version 2.0u63, release date December 1996). ALIGN compares entire sequences against one another, while LFASTA compares regions of local similarity. These alignment tools and their respective tutorials are available on the Internet at the NCSA Website, for instance. Alternatively, for comparisons of amino acid sequences of greater than about 30 amino acids, the Blast 2 sequences function can be employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). The BLAST sequence comparison system is available, for instance, from the NCBI web site; see also Altschul et al., J. Mol. Biol., 215:403-410, 1990; Gish. & States, Nature Genet., 3:266-272, 1993; Madden et al. Meth. Enzymol., 266:131-141, 1996; Altschul et al., Nucleic Acids Res., 25:3389-3402, 1997; and Zhang & Madden, Genome Res., 7:649-656, 1997.

Photoreceptor proteins to be used in the method of the present invention are not particularly limited and include any protein or protein fragment that is capable of undergoing a conformational change in response to absorption of photons of a particular wavelength, and, as a consequence, displays binding to a particular binding partner in a light-dependent manner.

In some embodiments, the photoreceptor protein is a phytochrome. As used herein, the term “phytochrome” has its general meaning in the art and refers to a family of photosensory molecules that plants and bacteria use to monitor informational light signals in the environment. These molecules, together with other informational photoreceptors, including the cryptochromes and phototropins provide plants and bacteria with the capacity to continuously track the presence, absence, spectral quality, fluence rate, directionality and diurnal duration of incoming light signals, and to adjust their growth and development toward optimal radiant energy capture, survival and reproduction.

In some embodiments, the phytochrome is selected from the group consisting of Phytochrome A (PhyA), Phytochrome B (PhyB), Phytochrome C (PhyC), Phytochrome D (PhyD), and Phytochrome E (PhyE).

In some embodiments, the photoreceptor is Phytochrome B (PhyB), and most preferably the first 651 amino-terminal amino acids of PhyB (i.e. HoloPhyB as set for in SEQ ID NO:2 or SEQ ID NO:3). In some embodiments, the photoreceptor protein can be PhyB derived from Arabidopsis thaliana. In some embodiments, the photoreceptor comprises an amino acid sequence that has at least 90% of identity with the amino acid sequence as set forth in SEQ ID NO: 2. In some embodiment, the photoreceptor comprises or refers to an amino acid sequence as set forth in SEQ ID NO:2 or SEQ ID NO:3.

SEQ ID NO: 2
MVSGVGGSGGGRGGGRGGEEEPSSSHTPNNRRGGEQAQSSGTKSLRPRS
NTESMSKAIQQYTVDARLHAVFEQSGESGKSFDYSQSLKTTTYGSSVPE
QQITAYLSRIQRGGYIQPFGCMIAVDESSFRIIGYSENAREMLGIMPQS
VPTLEKPEILAMGTDVRSLFTSSSSILLERAFVAREITLLNPVWIHSKN
TGKPFYAILHRIDVGVVIDLEPARTEDPALSIAGAVQSQKLAVRAISQL
QALPGGDIKLLCDTVVESVRDLTGYDRVMVYKFHEDEHGEVVAESKRDD
LEPYIGLHYPATDIPQASRFLFKQNRVRMIVDCNATPVLVVQDDRLTQS
MCLVGSTLRAPHGCHSQYMANMGSIASLAMAVIINGNEDDGSNVASGRS
SMRLWGLVVCHHTSSRCIPFPLRYACEFLMQAFGLQLNMELQLALQMSE
KRVLRTQTLLCDMLLRDSPAGIVTQSPSIMDLVKCDGAAFLYHGKYYPL
GVAPSEVQIKDVVEWLLANHADSTGLSTDSLGDAGYPGAAALGDAVCGM
AVAYITKRDFLFWFRSHTAKEIKWGGAKHHPEDKDDGQRMHPRSSFQAF
LEVVKSRSQPWETAEMDAIHSLQLILRDSFKESEAAMNSKVVDGVVQPC
RDMAGEQGIDELGAGTLEKLVDGAGSWSHPQFEKENLYFQGLEHHHHHH
SEQ ID NO: 3
MVSGVGGSGGGRGGGRGGEEEPSSSHTPNNRRGGEQAQSSGTKSLRPRS
NTESMSKAIQQYTVDARLHAVFEQSGESGKSFDYSQSLKTTTYGSSVPE
QQITAYLSRIQRGGYIQPFGCMIAVDESSFRIIGYSENAREMLGIMPQS
VPTLEKPEILAMGTDVRSLFTSSSSILLERAFVAREITLLNPVWIHSKN
TGKPFYAILHRIDVGVVIDLEPARTEDPALSIAGAVQSQKLAVRAISQL
QALPGGDIKLLCDTVVESVRDLTGYDRVMVYKFHEDEHGEVVAESKRDD
LEPYIGLHYPATDIPQASRFLFKQNRVRMIVDCNATPVLVVQDDRLTQS
MCLVGSTLRAPHGCHSQYMANMGSIASLAMAVIINGNEDDGSNVASGRS
SMRLWGLVVCHHTSSRCIPFPLRYACEFLMQAFGLQLNMELQLALQMSE
KRVLRTQTLLCDMLLRDSPAGIVTQSPSIMDLVKCDGAAFLYHGKYYPL
GVAPSEVQIKDVVEWLLANHADSTGLSTDSLGDAGYPGAAALGDAVCGM
AVAYITKRDFLFWFRSHTAKEIKWGGAKHHPEDKDDGQRMHPRSSFQAF
LEVVKSRSQPWETAEMDAIHSLQLILRDSFKESEAAMNSKVVDGVVQPC
RDMAGEQGIDELGAGSGSGLNDIFEAQKIEWHEHHHHHH

Thus in particular embodiment, the invention refers to a light-controlled molecular system comprising:
a. at least one recombinant protein comprising a variable domain of an antibody that is fused at its c-terminal end to PIF6, and
b. at least one tumor-antigen targeting antibody that is fused at its c-terminal end to Phytochrome B.

Thus in particular embodiment, the invention refers to a light-controlled molecular system comprising:
c. at least one recombinant protein comprising a variable domain of an antibody specific for TCR or CD3epsilon that is fused at its c-terminal end to PIF6, and
d. at least one tumor-antigen targeting single domain antibody that is fused at its c-terminal end to Phytochrome B.

In another embodiment, the compound that can interact with a photoactivable agent in a light-dependent manner is the photoactivable agent fused to antigen-tumor antibody, wherein the photoactivable agent can multimerize in a light-dependent manner.

In some embodiment, the photoactivable agent which can multimerize in a light-dependent manner is a photoreceptor protein which can dimerize in a light-dependent manner.

In some embodiment, the compound that can interact with a photoactivable agent in a light-dependent manner is a photoreceptor protein and the photoactivable agent fused to antigen-associated tumor antibody is the same photoreceptor protein, wherein the photoisomerizable protein can dimerize in a light-dependent manner.

Thus in particular embodiment, the invention refers to a light-controlled molecular system comprising:
a. at least one recombinant protein comprising a variable domain of an antibody that is fused at its c-terminal end to a photoreceptor protein which can dimerize in a light-dependent manner, and
b. at least one tumor-antigen targeting antibody that is fused at its c-terminal end to the photoreceptor protein.

Thus in particular embodiment, the invention refers to a light-controlled molecular system comprising:
a. at least one recombinant protein comprising a variable domain of an antibody specific for TCR beta or CD3epsilon that is fused at its c-terminal end to a photoreceptor protein which can dimerize in a light-dependent manner, and
b. at least one tumor-antigen targeting antibody that is fused at its c-terminal end to the photoreceptor protein.

In some embodiment, the photoreceptor protein which can dimerize in a light-dependent manner is a cryptochrome or phytochrome.

As used herein, the term “cryptochrome” has its general meaning in the art and refers to a family of photosensory molecules that belong to the flavoproteins superfamily. They are involved in the circadian rhythms and the sensing of magnetic fields in a number of species.

In some embodiments, the photoreceptor protein which can multimerize in a light-dependent manner is cryptochrome 1 (Cry1) or cryptochrome 2 (Cry2).

In some embodiments, the photoreceptor protein which can multimerize in a light-dependent manner is cryptochrome 2 (Cry2)

In some embodiments, the photoreceptor protein which can multimerize in a light-dependent manner can be Cry2 derived from Arabidopsis thaliana.

In some embodiment, the photoactivable agent which can dimerize in a light-dependent manner is a photoisomerizable compound which can dimerize in a light-dependent manner.

In some embodiment, the compound that can interact with a photoactivable agent in a light-dependent manner is a photoisomerizable compound and the photoactivable agent fused to antigen-associated tumor antibody is the same photoisomerizable compound, wherein the photoisomerizable protein can dimerize in a light-dependent manner.

Thus in particular embodiment, the invention refers to a light-controlled molecular system comprising:
a. at least one recombinant protein comprising a variable domain of an antibody that is fused at its c-terminal end to photoisomerizable compound which can dimerize in a light-dependent manner, and
b. at least one tumor-antigen targeting antibody that is fused at its c-terminal end to the same photoisomerizable compound.

As used herein, photoisomerizable compound has its general meaning in the art and refers to compounds subject to photoisomerism, i.e a form of isomerization induced by light. Photoisomerizable compound include but are not limited to azobenzenes, stilbenes, spiropyrans.

In some embodiment, the photoisomerizable compound is azobenzene or its derivatives.

As used herein, the term azobenzene refers to a photoswitchable chemical compound composed of two phenyl rings linked by a N═N double bond, which have the following formula:

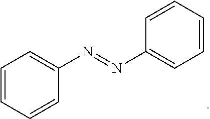 [SMILES: c1ccc(/N=N/c2ccccc2)cc1]

Azobenzene derivatives includes maleimide azobenzene maleimide, dioxane-methoxy-azobenzenes and tetrachloro-azobenzenes.

In some embodiment, the photoisomerizable compound is azobenzene-based coiled coil domain or azobenzene derivative-based coiled coil domain, such as described in Fuzhong Zhang et al. Angew Chem Int Ed Engl 49, 2010.

A further aspect of the present invention relates to a nucleic acid encoding for the light-controlled molecular system of the present invention. As used herein, the term “nucleic acid molecule” has its general meaning in the art and refers to a DNA or RNA molecule. However, the term captures sequences that include any of the known base analogues of DNA and RNA such as, but not limited to 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyamino-methyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil,-uracil-5-oxyacetic acid methylester, uracil-5-oxyacetic acid, pseudouracil, queosine, 2-thiocytosine, and 2,6-diaminopurine. In some embodiments, the nucleic acid molecule of the present invention is included in a suitable vector, such as a plasmid, cosmid, episome, artificial chromosome, phage or a viral vector. So, a further object of the invention relates to a vector comprising a nucleic acid encoding for the light-controlled molecular system of the present invention. Typically, the vector is a viral vector which is an adeno-associated virus (AAV), a retrovirus, bovine papilloma virus, an adenovirus vector, a lentiviral vector, a vaccinia virus, a polyoma virus, or an infective virus. In some embodiments, the vector is an AAV vector.

A further object of the present invention relates to a host cell transformed with the nucleic acid molecule of the present invention. The term “transformation” means the introduction of a “foreign” (i.e. extrinsic or extracellular) gene, DNA or RNA sequence to a host cell, so that the host cell will express the introduced gene or sequence to produce a desired substance, typically a protein or enzyme coded by the introduced gene or sequence. A host cell that receives and expresses introduced DNA or RNA has been “transformed”. For instance, as disclosed above, for expressing and producing the polypeptide of the present invention, prokaryotic cells and, in particular E. coli cells, will be chosen. Actually, according to the invention, it is not mandatory to produce the light-controlled molecular system of the present invention in a eukaryotic context that will favour post-translational modifications (e.g. glycosylation). Typically, the host cell may be suitable for producing the polypeptide of the present invention as described above. In some embodiments, the host cells is isolated from a mammalian subject who is selected from a group consisting of: a human, a horse, a dog, a cat, a mouse, a rat, a cow and a sheep. In some embodiments, the host cell is a human cell. In some embodiments, the host cell is a cell in culture. The cells may be obtained directly from a mammal (preferably human), or from a commercial source, or from tissue, or in the form for instance of cultured cells, prepared on site or purchased from a commercial cell source and the like. In some embodiments, the host cell is a mammalian cell line (e.g., Vero cells, CHO cells, 3T3 cells, COS cells, etc.).

### Method of Activating on Demand an Immune Cell or a Plurality of Immune Cells of the Invention

Accordingly, another object of the present invention relates to a method of activating on demand an immune cell or a plurality of immune cells comprising:
i) contacting the immune cell or the plurality of immune cells with the light-controlled molecular system of the invention as described above,
ii) exposing the cell or the plurality of cells with a suitable wavelength of light wherein said exposition allows the oligomerization of a complex binding the recombinant protein of said system to the tumor-associated antigen antibody of said system and thus triggering the activation of the immune cell or plurality of immune cells to lyse tumor cell.

In other words, the invention refers to the light-controlled molecular system of the invention for use for activating on demand an immune cell or a plurality of immune cells.

In particular embodiment the invention refers to the light-controlled molecular system of the invention for use for activating on demand an immune cell or a plurality of immune cells comprising:
i) contacting the immune cell or the plurality of immune cells with said light-controlled molecular system, and
ii) exposing the cell or the plurality of cells with a suitable wavelength of light wherein said exposition allows the oligomerization of a complex binding the recombinant protein of said light-controlled molecular system to the tumor-associated antigen antibody of said light-controlled molecular system and thus triggering the activation of the immune cell or plurality of immune cells to lyse tumor cell.

In other words, the present invention relates to a method of activating on demand an immune cell or a plurality of immune cells comprising:
i) contacting the immune cell or the plurality of immune cells with (a) at least one recombinant protein comprising a variable domain of an antibody that is fused at its c-terminal end to a compound that can interact with a photoactivable agent in a light-dependent manner, and (b) at least one tumor-antigen targeting antibody that is fused at its c-terminal end to the photoactivable agent,
ii) exposing the cell or the plurality of cells with a suitable wavelength of light wherein said exposition allows the oligomerization of a complex binding the recombinant protein to the tumor-associated antigen antibody and thus triggering the activation of the immune cell or plurality of immune cells to lyse tumor cell.

Thus, in some embodiment, the present invention relates to a method of activating on demand an immune cell or a plurality of immune cells comprising:
i) contacting the immune cell or the plurality of immune cells with (a) at least one recombinant protein comprising a variable domain of an antibody that is fused at its c-terminal end to a factor that can interact with a photoreceptor protein in a light-dependent manner, and (b) at least one tumor-antigen targeting antibody that is fused at its c-terminal end to the photoreceptor protein; and
ii) exposing the cell or the plurality of cells with a suitable wavelength of light wherein said exposition allows the oligomerization of a complex binding the recombinant protein to the tumor-antigen antibody and thus triggering the activation of the immune cell or plurality of immune cells to lyse tumor cell.

Thus, in some embodiment, the present invention relates to a method of activating on demand an immune cell or a plurality of immune cells comprising:
i) contacting the immune cell or the plurality of immune cells with (a) at least one recombinant protein comprising a variable domain of an antibody that is fused at its c-terminal end to PIF6, and (b) at least one tumor-antigen targeting antibody that is fused at its c-terminal end to PhytochromeB; and
ii) exposing the cell or the plurality of cells with a suitable wavelength of light wherein said exposition allows the oligomerization of a complex binding the recombinant protein to the tumor-antigen antibody and thus triggering the activation of the immune cell or plurality of immune cells to lyse tumor cell.

Thus, in some embodiment, the present invention relates to a method of activating on demand an immune cell or a plurality of immune cells comprising:
i) contacting the immune cell or the plurality of immune cells with (a) at least one recombinant protein comprising a variable domain of an antibody that is fused at its c-terminal end to a photoreceptor protein which can dimerize in a light-dependent manner, and (b) at least one tumor-associated antigen targeting antibody that is fused at its c-terminal end to the photoreceptor protein; and
ii) exposing the cell or the plurality of cells with a suitable wavelength of light wherein said exposition allows the oligomerization of a complex binding the recombinant protein to the tumor-antigen antibody and thus triggering the activation of the immune cell or plurality of immune cells to lyse tumor cell.

In some embodiment, the variable domain comprised in the recombinant protein is a single domain antibody, and more particularly an agonistic single domain antibody specific for a TCR Beta or CD3epsilon.

In some embodiments, the recombinant protein of the present invention comprises a Fab fragment derived from an agonistic antibody specific for a TCR Beta or CD3epsilon wherein the VH domain of the Fab fragment is fused at its c-terminal end to a factor that can interact with a photoreceptor protein in a light-dependent manner.

In some embodiment, the tumor-associated antigen targeting antibody is a single domain antibody.

In some embodiment, the photoreceptor protein which can dimerize in a light-dependent manner is a cryptochrome or phytochrome.

As used herein, the term “cryptochrome” has its general meaning in the art and refers to a family of photosensory molecules that belong to the flavoproteins superfamily. They are involved in the circadian rhythms and the sensing of magnetic fields in a number of species.

In some embodiments, the photoreceptor protein which can multimerize in a light-dependent manner is cryptochrome 1 (Cry1) or cryptochrome 2 (Cry2).

In some embodiments, the photoreceptor protein which can multimerize in a light-dependent manner is cryptochrome 2 (Cry2)

In some embodiments, the photoreceptor protein which can multimerize in a light-dependent manner can be Cry2 derived from Arabidopsis thaliana.

In some embodiment, the photoactivable agent which can dimerize in a light-dependent manner is a photoisomerizable compound which can dimerize in a light-dependent manner.

In some embodiment, the compound that can interact with a photoactivable agent in a light-dependent manner is a photoisomerizable compound and the photoactivable agent fused to antigen-associated tumor antibody is the same photoisomerizable compound, wherein the photoisomerizable protein can dimerize in a light-dependent manner.

Thus, in some embodiment, the present invention relates to a method of activating on demand an immune cell or a plurality of immune cells comprising:
i) contacting the immune cell or the plurality of immune cells with (a) at least one recombinant protein comprising a variable domain of an antibody that is fused at its c-terminal end to a photoisomerizable compound which can dimerize in a light-dependent manner, and (b) at least one tumor-associated antigen targeting antibody that is fused at its c-terminal end to the same photoisomerizable compound; and
ii) exposing the cell or the plurality of cells with a suitable wavelength of light wherein said exposition allows the oligomerization of a complex binding the recombinant protein to the tumor-antigen antibody and thus triggering the activation of the immune cell or plurality of immune cells to lyse tumor cell.

In some embodiments, the method further comprises a step of interrupting the activation on demand of the immune cell or the plurality of immune cells by exposing with a suitable wavelength of light wherein said exposition blocks the oligomerization of the recombinant protein and thus interrupts the activation of the immune cell or plurality of immune cells.

As used herein, the term “on-demand” refers to fact that the operator control finely and immediately the activation or deactivation of the immune cell or plurality of immune cells.

Suitable wavelengths of light for the activation or deactivation of the photoreceptor protein, are known in the art for each particular photoreceptor protein. As an example, in case the photoreceptor protein is a phytochrome, activation can be effected by light having a wavelength of between 500 and 720 nm, preferably between 620 and 700 nm, and most preferably of about 650 nm. Further, deactivation can be effect by light having a wavelength of more than 720 nm, preferably of about 750 nm.

Suitable wavelengths of light for the activation or deactivation of the photoisomerizable compound, are known in the art for each particular photoisomerizable compound. As an example, in case the photoisomerizable compound is an azobenzenes, activation can be effected by light having a wavelength of between 250 and 390 nm, preferably between 320 and 390 nm, and most preferably of about 360 nm. Further, deactivation can be effect by light having a wavelength of more than 420 nm, preferably of about 450 nm.

In some embodiments, the immune cell or the plurality of immune cells are exposed with continuous or pulsatile suitable wavelength of light.

As used herein, the term “continuous exposition” has its general meaning in the art and refers to a constant stimulation with a suitable wavelength of light

In some embodiments, the immune cell or the plurality of immune cells are exposed with short-pulsed suitable wavelength of light.

As used herein, the term “immune cell” includes cells that are of hematopoietic origin and that play a role in the immune response. Immune cells include cells of the innate immune system and cells of the adaptive immune system. Immune cells include, for example, lymphocytes, such as B cells and T cells; natural killer cells; and myeloid cells, such as monocytes, macrophages, eosinophils, mast cells, basophils, and granulocytes.

In some embodiments, the immune cell is a cell of the innate immune system. The “innate immune system” is the nonspecific immune system that controls the body's response to an agent until the more specific adaptive immune system can produce specific antibodies and/or T cells (Modlin et al, N. Engl. J. Med 1999, 340:1834-1835). The innate immune system generally involves phagocytic cells (e.g., neutrophils, monocytes, and macrophages); cells that release inflammatory mediators (e.g., basophils, mast cells, and eosinophils); natural killer cells (NK cells); and dendritic cells (DCs). In contrast, the “adaptive”, or “acquired, immune system”, is very specific in its responses. It is called an adaptive system because is occurs during the lifetime of an individual as an adaptation to infection with a pathogen. Adaptive immunity can be artificially acquired in response to a vaccine (antigens) or by administering antibodies, or can be naturally acquired by infection.

In some embodiments, the immune cell is an antigen presenting cell. As used herein, “antigen presenting cell” refers to cells that display foreign antigens complexed with major histocompatibility complexes (MHCs) on their surfaces, which are then recognized by T cells using their T cell receptors. Antigen presenting cells include cells that constitutively express MHC molecules (e.g., B lymphocytes, monocytes, dendritic cells, and Langerhans cells) as well as other antigen presenting cells that do not constitutively express MHC molecules (e.g., keratinocytes, endothelial cells, astrocytes, fibroblasts, and oligodendrocytes).

In some embodiments, the immune cell is a T cell. As used herein, the term “T cell” (i.e., T lymphocyte) is intended to include all cells within the T cell lineage, including thymocytes, immature T cells, mature T cells and the like, from a mammal (e.g., human). T cells include mature T cells that express either CD4 or CD8, but not both, and a T cell receptor. The various T cell populations described herein can be defined based on their cytokine profiles and their function. As used herein, the term “naive T cells” includes T cells that have not been exposed to cognate antigen and so are not activated or memory cells. Naive T cells are not cycling and human naive T cells are CD45RA+. If naive T cells recognize antigen and receive additional signals depending upon but not limited to the amount of antigen, route of administration and timing of administration, they may proliferate and differentiate into various subsets of T cells, e.g., effector T cells. As used herein, the term “effector T cell” includes T cells which function to eliminate antigen (e.g., by producing cytokines which modulate the activation of other cells or by cytotoxic activity). The term “effector T cell” includes T helper cells (e.g., Th1 and Th2 cells) and cytotoxic T cells. Th1 cells mediate delayed type hypersensitivity responses and macrophage activation while Th2 cells provide help to B cells and are critical in the allergic response (Mosmann and Coffman, 1989, Anna. Rev. Immunol. 7, 145-173; Paul and Seder, 1994, Cell 76, 241-251; Arthur and Mason, 1986, J. Exp. Med. 163, 774-786; Paliard et al., 1988, J. Immunol. 141, 849-855; Finkelman et al., 1988, J. Immunol. 141, 2335-2341). As used herein, the term “regulatory T cell” includes T cells which produce low levels of IL-2, IL-4, IL-5, and IL-12. Regulatory T cells produce TNFa, TGFp, IFN-γ, and IL-10, albeit at lower levels than effector T cells. Although TGFP is the predominant cytokine produced by regulatory T cells, the cytokine is produced at lower levels than in Th1 or Th2 cells, e.g., an order of magnitude less than in Th1 or Th2 cells. Regulatory T cells can be found in the CD4+CD25+ population of cells (see, e.g., Waldmann and Cobbold. 2001. Immunity. 14:399). Regulatory T cells actively suppress the proliferation and cytokine production of Th1, Th2, or naive T cells which have been stimulated in culture with an activating signal (e.g., antigen and antigen presenting cells or with a signal that mimics antigen in the context of MHC, e.g., anti-CD3 antibody plus anti-CD28 antibody). As used herein, the term “exhausted T cell” refers to malfunctional T cells that are characterized by the stepwise and progressive loss of T-cell functions and can culminate in the physical deletion of the responding cells. Exhausted T cell may arise during chronic infections and cancer. For example, exhaustion is well-defined during chronic lymphocytic choriomeningitis virus infection and commonly develops under conditions of antigen-persistence, which occur following many chronic infections that are of significant public health concern including hepatitis B virus, hepatitis C virus and human immunodeficiency virus infections, as well as during tumor outgrowth (see. e.g., John Wherry, Nature Immunology 12, 492-499, 2011). As used herein, the term “anergic T cell” refers to T cells that are functionally inactivated and unable to initiate a productive response even when antigen is encountered in the presence of full co-stimulation (see, e.g., Macian F. et al, Curr Opin Immunol. 2004, 16 (2): 209-16.) T cell anergy is a tolerance mechanism in which the lymphocyte is intrinsically functionally inactivated following an antigen encounter, but remains alive for an extended period of time in a hyporesponsive state. Models of T cell anergy affecting both CD4+ and CD8+ cells fall into two broad categories. One, clonal anergy, is principally a growth arrest state, whereas the other, adaptive tolerance or in vivo anergy, represents a more generalized inhibition of proliferation and effector functions (see, e.g., Schwartz RH. Annu Rev Immunol. 2003; 21:305-34).

In some embodiments, the plurality of cells is encompasses in a tissue, an organ or an organism. Accordingly, the method of the present invention can be applied to in vitro or in vivo system.

In some embodiment, the method of activating on demand an immune cell or a plurality of immune cells is an in vitro method.

In some embodiment, the light-controlled molecular system of the invention is contacted with the immune cell or the plurality of immune cell by administering the light molecular system in the tissue, the organ or the organism encompassing the immune cell or the plurality of immune cell.

Thus, in some embodiment, the invention refers to a method of activating on demand an immune cell or a plurality of immune cells in a subject comprising:
i. administering to said subject the light-controlled molecular c of the invention, and
ii. exposing the cell or the plurality of immune cells with a suitable wavelength of light wherein said exposition allows the oligomerization of a complex binding the recombinant protein of the light-controlled molecular system to the tumor-antigen antibody of the light-controlled molecular system.

As used herein, the term “tissue” as used herein refers to any type of tissue in human or animals, and includes, but is not limited to, vascular tissue, skin tissue, hepatic tissue, pancreatic tissue, neural tissue, urogenital tissue, gastrointestinal tissue, skeletal tissue including bone and cartilage, adipose tissue, connective tissue including tendons and ligaments, amniotic tissue, chorionic tissue, dura, pericardia, muscle tissue, glandular tissue, facial tissue, ophthalmic tissue.

In some embodiments, the tissue is a tumor tissue, and more particularly a solid tumor tissue.

As used herein, the term “tumor tissue” means both tissue known to contain a tumor and tissue believed to contain a tumor. Here, the term “tumor” comprises both benign tumors and malignant tumors. In particular, the term “tumor” comprises cancers and, in particular, metastasizing cancers and carcinomas. Here, the term “tumor” comprises solid tumor tissue and liquid tumor tissue.

In some embodiment, the tumor is a cancer.

As used herein, the term “cancer” refers to an abnormal cell having the capacity for autonomous growth, i.e., an abnormal state or condition characterized by rapidly proliferating cell growth with the potential to invade or spread to other parts of the body. The term is meant to include all types of cancerous growths or oncogenic processes, metastatic tissues or malignantly transformed cells, tissues, or organs, irrespective of histopathologic type or stage of invasiveness. The terms “cancer” or “neoplasms” include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, glioblastoma non-small cell carcinoma of the lung, cancer of the small intestine and cancer of the esophagus.

As used herein, the term “cancer” has its general meaning in the art and includes, but is not limited to, solid tumors and blood borne tumors. The term cancer includes diseases of the skin, tissues, organs, bone, cartilage, blood and vessels. The term “cancer” further encompasses both primary and metastatic cancers. Examples of cancers include, but are not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus.

In particular embodiment, the cancer is selected from the group consisting of, but not limited to, head and neck squamous cell carcinoma (HNSCC); adrenal cortical cancer; anal cancer; periphilar cancer; distal bile duct cancer; intrahepatic bile duct cancer; osteoblastoma; osteochrondroma; hemangioma; chondromyxoid fibroma; astrocytoma; ductal carcinoma in situ; gynecomastia; endometrial adenocarcinoma; adenocanthoma; papillary serous adenocarcinoma; laryngeal and hypopharyngeal cancer; hemangioma, hepatic adenoma; focal nodular hyperplasia; small cell lung cancer; non-small cell lung cancer; mesothelioma, plasmacytoma; esthesioneuroblastoma; midline granuloma; nasopharyngeal cancer; oral cavity and oropharyngeal cancer, ovarian cancer; pancreatic cancer; penile cancer; pituitary cancer; prostate cancer; salivary gland cancer; non-melanoma skin cancer; stomach cancer, testicular cancer; thymus cancer; follicular carcinoma; anaplastic carcinoma; poorly differentiated carcinoma; medullary thyroid carcinoma; vaginal cancer, vulvar cancer, uterine leiomyosarcoma; bladderneoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating ductal carcinoma; medullary carcinoma; infiltrating lobular carcinoma; lobular carcinoma in situ; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; leydig cell tumor, malignant; lipid cell tumor, malignant; paraganglioma, malignant; gliomas; medulloblastoma; Schwannoma; germinoma; craniopharyngioma; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; uveal melanoma, intraocular lymphoma, conjunctival tumor, lacrimal gland tumors, blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; chorioadenoma destruens; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiment, the cancer is skin cancer.

In some embodiment, the cancer is ocular cancer.

In some embodiment, the cancer is melanoma cancer.

In some embodiment, the cancer is a solid cancer.

In some embodiment, the cancer is coloncarcinoma or bladder cancer.

As used herein the term “organ” refers to a solid vascularized organ that performs a specific function or group of functions within an organism. The term organ includes, but is not limited to heart, lung, kidney, liver, pancreas, skin, uterus, bone, cartilage, small or large bowel, bladder, brain, breast, blood vessels, esophagus, fallopian tube, gallbladder, ovaries, pancreas, prostate, placenta, spinal cord, limb including upper and lower, spleen, stomach, testes, thymus, thyroid, trachea, ureter, urethra, uterus.

As used herein, the term “organism” refers to any living creature capable of reproduction. In some embodiments, the organism is a mammal. The term “mammal” refers preferably, but is not limited to, to such organisms as rodents, ungulates, primates, mice, rats, rabbits, guinea pigs, horses, sheep, pigs, goats, and cows, more preferably to cats, dogs, monkeys, and apes, and most preferably to humans.

In some embodiments, the intensity of light to which the immune cell or the plurality of immune cells is exposed can be used to control the extent of the activation. For example, low-intensity red light will achieve only partial, titrated association. Total illumination doses less than 1,000 micromoles of photons per square meter can be regarded as low intensity red light. Total illumination doses greater than 10,000 micromoles of photons per square meter can be regarded as high-intensity light that is sufficient for 100% conversion. The intensity of red light required to convert a significant fraction or majority or substantially all the photoreceptor to an activated state can be empirically.

In some embodiments, the time of exposure to light can be varied according to effect needed and light intensity chosen, e.g., for about 1, 10 or 100 milliseconds, or about 1, 5 or 10 seconds, or about 1, 2, 3, 5, 10, 20 or 30 minutes, or about 1, 2, 3 or 5 hours, or about 1, 2, 3, or 5 days, or 1, 2 or 3 weeks. In some embodiments, the cell or plurality of cells is/are exposed for a short time. For example, the cell or plurality of cells can be exposed to ref or infra-red light for less than a minute, e.g., about 1, 5, 10, 20 or 40 seconds. The light can be delivered by known devices such as a laser, or led in one or more pulses or individual portions. For example, a UV-pumped red dye cell laser or red led can shoot ultrafast pulses of light that last about 5 ns; these can be applied, e.g., at low intensity at about 20 Hz for about 5 s to minutes.

In some embodiments, the immune cell or the plurality of immune cells are exposed with continuous or pulsatile suitable wavelength of light.

As used herein, the term “continuous exposition” has its general meaning in the art and refers to a constant exposure with a suitable wavelength of light.

As used herein, the term “pulsatile exposition” has its general meaning in the art and refers to a pulsed exposure with a suitable wavelength of light, i.e a repetition of exposure by a suitable wavelength of light following by no exposure.

In some embodiments, the immune cell or the plurality of immune cells are exposed with short-pulsed suitable wavelength of light.

The method of the present invention allows modulating temporarily the activation of the immune cell or plurality of immune cells against the tumor cell. The method disclosed herein can indeed allow extremely quick activation of the immune cell or plurality of immune cells. Accordingly, the method of the present invention allows control of activation of the immune cell or the plurality of immune cells within 1 minute, or sometimes within 10-20 seconds, and sometimes even within one second.

The method of the present invention also allow modulation spatially the activation of the immune cell or plurality of immune cells. Said activation can be locally triggered especially and thus can be restricted to a particular tissue, organ or organism. For example a portion of a tissue, organ or organism can be exposed to “activating” light (such as activating red light) that induces the formation of a complex binding the recombinant protein to the tumor-antigen antibody, and thus induces the tumor cell killing by cytotoxic T lymphocyte cell (CTL). In another example, the tissue, organ or organism can be bathed in continuous “inactivating” light (and in particular in “inactivating” infrared light), while a localized beam of activating light (and in particular of activating red light) is restrictively delivered to a specific portion the tissue, organ or organism, resulting in well-defined localization.

### Method for Treating Cancer of the Invention

The method of the present invention is suitable to treat cancer in a subject in need thereof.

A further object of the present invention relates to a method for treating tumor in a subject in need thereof, comprising:
i. administering to said subject a therapeutically effective amount of the light-controlled molecular system of the invention; and
ii. exposing the tumor with a suitable wavelength of light wherein said exposition allows the formation of a complex binding the recombinant protein of the light-controlled molecular system of the invention to the tumor-antigen antibody of the light-controlled molecular system of the invention.

Thus, the invention refers to the light-controlled molecular system of the invention for use as a medicament.

In particular embodiment, the invention refers to the light-controlled molecular system of the invention for use for treating tumor in a subject in need thereof.

In other words, the present invention relates to a relates to a method for treating tumor in a subject in need thereof, comprising:
i. administering to said subject a therapeutically effective amount of (a) at least one recombinant protein comprising a variable domain of an antibody that is fused at its c-terminal end to a compound that can interact with a photoactivable agent in a light-dependent manner, and a therapeutically effective amount of (b) at least one tumor-antigen targeting antibody that is fused at its c-terminal end to the photoactivable agent; and
ii. exposing the tumor with a suitable wavelength of light wherein said exposition allows the formation of a complex binding the recombinant protein to the tumor-antigen antibody.

In some embodiment, the tumor is cancer.

In some embodiment, the cancer is skin cancer, ocular cancer, breast cancer or colon cancer.

In some embodiment, the tumor is skin tumor or ocular tumor.

In some embodiment, the cancer is melanoma cancer.

In some embodiment, the cancer is carcinoma cancer.

In some embodiment, the cancer is a solid cancer.

In particular embodiment, the tumor is exposed with a suitable wavelength of light via an external light (i.e the light source is outside of body of the subject).

In some embodiment, the compound that can interact with a photoactivable agent in a light-dependent manner is a factor that can interact with a photoreceptor protein in a light-dependent manner and the photoactivable agent fused to antigen-associated tumor antibody is the photoreceptor protein.

In some embodiment, the compound that can interact with a photoactivable agent in a light-dependent manner is a photoreceptor protein and the photoactivable agent fused to antigen-associated tumor antibody is the same photoreceptor protein, wherein the photoreceptor proteins can dimerize in a light-dependent manner.

In some embodiment, the tumor-antigen targeting antibody is a single domain antibody.

In some embodiments, when the method treat melanoma cancer, the tumor-antigen targeting antibody is specific for TRP1 (TRP1-targeting antibody) or EpCAM (EpCAM-targeting antibody).

In some embodiments, the Fab fragment comprising in the recombinant protein derives from an agonistic antibody whose the monovalent form is not able to induce the biological signaling activity of the receptor.

In some embodiments, the agonistic antibody is specific for a receptor of an immune cell

In some embodiments, the agonistic antibody is specific for a TCR.

In some embodiments, the agonistic antibody is specific for TCR Beta or CD3epsilon.

In some embodiment, the tumor-antigen targeting antibody is conjugated to the photoactivable agent by any suitable means, as will be apparent to those of skill in the art. In some embodiments, the variable domain and the compound that can interact with a photoactivable agent in a light-dependent manner are fused to each other by any suitable means, as will be apparent to those of skill in the art. In some embodiments, the tumor-antigen targeting antibody and the photoactivable agent are fused to each other directly or via a linker. In some embodiments, the variable domain and the factor that can interact with a photoreceptor protein in a light-dependent manner are fused to each other directly (i.e. without use of a linker) or via a linker. In some embodiment, the click chemistry can be used to conjugated the tumor-antigen antibody to the photoactivable agent and/or to fused the variable domain and the compound that can interact with a photoactivable agent in a light-dependent manner.

In some embodiments, the tumor-antigen targeting antibody is conjugated with biotin, and fused to the biotinylated photoreceptor protein via streptavidin, avidin or neutravidin.

In some embodiments, at least two tumor-antigen targeting antibody that is fused at its c-terminal end to the photoactivable agent are administered in the method of the present invention.

In some embodiments, two tumor-antigen targeting antibody are conjugated with biotin, and fused each to one photoactivable agent via streptavidin, avidin or neutravidin in order to form a complex composed of one streptavidin, avidin or neutravidin, two biotinylated-tumor-antigen targeting antibody and two biotinylated photoactivable agent as described in FIG. 1A.

As used herein the terms “administering” or “administration” refer to the act of injecting or otherwise physically delivering a substance as it exists outside the body into the subject, such as by parenteral, topical, in-situ, intraocular (intravitreal, intracameral, subconjunctival) mucosal, intradermal, intravenous, subcutaneous, percutaneous, intramuscular delivery and/or any other method of physical delivery described herein or known in the art. When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof.

In some embodiments, the light-controlled molecular system of the invention is administered into the tumor and the tumor-microenvironment.

In some embodiments, the light-controlled molecular system of the invention is administered intratumorally.

In some embodiments, the light-controlled molecular system of the invention is administered intravenously or subcutaneously.

As used herein, the term “treatment” or “treat” refer to both prophylactic or preventive treatment as well as curative or disease modifying treatment, including treatment of subjects at risk of contracting the disease or suspected to have contracted the disease as well as subjects who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

A “therapeutically effective amount” is intended for a minimal amount of active agent which is necessary to impart therapeutic benefit to a subject. For example, a “therapeutically effective amount” to a subject is such an amount which induces, ameliorates or otherwise causes an improvement in the pathological symptoms, disease progression or physiological conditions associated with or resistance to succumbing to a disorder. It will be understood that the total daily usage of the compounds of the present invention will be decided by the attending physician within the scope of sound medical judgment.

In some embodiment, the light-controlled molecular system of the invention can be administered in combination with anti-cancer therapy.

In some embodiment, the (a) at least one recombinant protein comprising a variable domain of an antibody that is fused at its c-terminal end to a compound that can interact with a photoactivable agent in a light-dependent manner, and the (b) at least one tumor-antigen targeting antibody that is fused at its c-terminal end to the photoactivable agent can be administered in combination with anti-cancer therapy.

As used herein, the term “anti-cancer therapy” has its general meaning in the art and refers to any compound, natural or synthetic, used for the treatment of cancer.

In a particular embodiment, the classical treatment refers to radiation therapy, antibody therapy or chemotherapy.

As used herein, the term “chemotherapeutic agent” refers to chemical compounds that are effective in inhibiting tumor growth. Examples of chemotherapeutic agents include multkinase inhibitors such as sorafenib and sunitinib, alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaorarnide and trimethylolomelamine; acetogenins (especially bullatacin and bullatacinone); a carnptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estrarnustine, ifosfamide, mechlorethamine, mechlorethamine oxide hydrochloride, melphalan, novembichin, phenesterine, prednimus tine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin (11 and calicheamicin 211, see, e.g., Agnew Chem Intl. Ed. Engl. 33:183-186 (1994); dynemicin, including dynemicin A; an esperamicin; as well as neocarzinostatin chromophore and related chromoprotein enediyne antiobiotic chromomophores), aclacinomysins, actinomycin, authramycin, azaserine, bleomycins, cactinomycin, carabicin, canninomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idanrbicin, marcellomycin, mitomycins, mycophenolic acid, nogalarnycin, olivomycins, peplomycin, potfiromycin, puromycin, quelamycin, rodorubicin, streptomgrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophospharnide glycoside; aminolevulinic acid; amsacrine; bestrabucil; bisantrene; edatraxate; defo famine; demecolcine; diaziquone; elfornithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pento statin; phenamet; pirarubicin; podophyllinic acid; 2-ethylhydrazide; procarbazine; PSK®; razoxane; rhizoxin; sizofiran; spirogennanium; tenuazonic acid; triaziquone; 2,2′,2″-trichlorotriethylarnine; trichothecenes (especially T-2 toxin, verracurin A, roridinA and anguidine); urethan; vindesine; dacarbazine; mannomustine; mitobromtol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.]) and doxetaxel (TAXOTERER, Rhone-Poulenc Rorer, Antony, France); chlorambucil; gemcitabine; 6-thioguanine; mercaptopurine; methotrexate; platinum analogs such as cisp latin and carbop latin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-1 1; topoisomerase inhibitor RFS 2000; difluoromethylornithine (DMFO); retinoic acid; capecitabine; and pharmaceutically acceptable salts, acids or derivatives of any of the above. Also included in this definition are antihormonal agents that act to regulate or inhibit honnone action on tumors such as anti-estrogens including for example tamoxifen, raloxifene, aromatase inhibiting 4 (5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

As used herein, the term “radiation therapy” has its general meaning in the art and refers the treatment of cancer with ionizing radiation. Ionizing radiation deposits energy that injures or destroys cells in the area being treated (the target tissue) by damaging their genetic material, making it impossible for these cells to continue to grow. One type of radiation therapy commonly used involves photons, e.g. X-rays. Depending on the amount of energy they possess, the rays can be used to destroy cancer cells on the surface of or deeper in the body. The higher the energy of the x-ray beam, the deeper the x-rays can go into the target tissue. Linear accelerators and betatrons produce x-rays of increasingly greater energy. The use of machines to focus radiation (such as x-rays) on a cancer site is called external beam radiation therapy. Gamma rays are another form of photons used in radiation therapy. Gamma rays are produced spontaneously as certain elements (such as radium, uranium, and cobalt 60) release radiation as they decompose, or decay. In some embodiments, the radiation therapy is external radiation therapy. Examples of external radiation therapy include, but are not limited to, conventional external beam radiation therapy; three-dimensional conformal radiation therapy (3D-CRT), which delivers shaped beams to closely fit the shape of a tumor from different directions; intensity modulated radiation therapy (IMRT), e.g., helical tomotherapy, which shapes the radiation beams to closely fit the shape of a tumor and also alters the radiation dose according to the shape of the tumor; conformal proton beam radiation therapy; image-guided radiation therapy (IGRT), which combines scanning and radiation technologies to provide real time images of a tumor to guide the radiation treatment; intraoperative radiation therapy (IORT), which delivers radiation directly to a tumor during surgery; stereotactic radiosurgery, which delivers a large, precise radiation dose to a small tumor area in a single session; hyperfractionated radiation therapy, e.g., continuous hyperfractionated accelerated radiation therapy (CHART), in which more than one treatment (fraction) of radiation therapy are given to a subject per day; and hypofractionated radiation therapy, in which larger doses of radiation therapy per fraction is given but fewer fractions.

As used herein, the term “immune checkpoint inhibitor” refers to molecules that totally or partially reduce, inhibit, interfere with or modulate one or more immune checkpoint proteins.

As used herein, the term “immune checkpoint protein” has its general meaning in the art and refers to a molecule that is expressed by T cells in that either turn up a signal (stimulatory checkpoint molecules) or turn down a signal (inhibitory checkpoint molecules).

Examples of stimulatory checkpoint include CD27 CD28 CD40, CD122, CD137, OX40, GITR, and ICOS. Examples of inhibitory checkpoint molecules include A2AR, B7-H3, B7-H4, BTLA, CTLA-4, CD277, IDO, KIR, PD-1, PD-L1, LAG-3, TIM-3 and VISTA.

The compounds used in connection with the treatment methods of the present invention are administered and dosed in accordance with good medical practice, taking into account the clinical condition of the individual subject, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement including, but not limited to, improved survival rate or more rapid recovery, or improvement or elimination of symptoms and other indicators as are selected as appropriate measures by those skilled in the art.

Any therapeutic agent of the invention may be combined with pharmaceutically acceptable excipients, and optionally sustained-release matrices, such as biodegradable polymers, to form therapeutic compositions.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The form of the pharmaceutical compositions, the route of administration, the dosage and the regimen naturally depend upon the condition to be treated, the severity of the illness, the age, weight, and sex of the patient, etc. The pharmaceutical compositions of the invention can be formulated for a topical, oral, intranasal, parenteral, intraocular, intravenous, intramuscular or subcutaneous administration and the like. Particularly, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The doses used for the administration can be adapted as a function of various parameters, and in particular as a function of the mode of administration used, of the relevant pathology, or alternatively of the desired duration of treatment. In addition, other pharmaceutically acceptable forms include, e.g. tablets or other solids for oral administration; time release capsules; and any other form currently can be used.

The invention will be further illustrated by the following figures and examples. However, these examples and figures should not be interpreted in any way as limiting the scope of the present invention.

### FIGURES

FIG. 1. Illustration of the Light-inducible Bispecific T cell Engager, an OptoFab system derivative for light driven tumor cell killing.

A. HoloPhyB is associated in a molecular complex to the melanoma cell targeting antibody TA-99 (specific for TRP-1 surface molecule). We named this complex Melanoma-targeted PhyB (Mt-PhyB). B. Mt-PhyB bind to the melanoma cells. In presence of Effector CD8 T cells loaded with the OptoFab, a red light exposure induced the capture of the OptoFab at the surface of the melanoma cell, engaging the TCR of effector CD8 T cells and triggering tumor cell killing.

FIG. 2. the Light-inducible Bispecific T cell Engager triggers tumor cell killing by CD8 effector T cells in response to light.

A. Mt-PhyB complex generation: upper panel, biotynilated HoloPhyB was added to streptavidin with 2:1 as ratio. The complexes PhyB-SA were analyzed by HPLC at and detected by following the absorbance at 280 nm and 680 nm (characterisitc for PhyB). Lower panel, an excess of biotinylated TA-99 Ab was added to the PhyB-SA complexes. The new complexes analysis by HPLC revealed the appearance of three new peaks of high molecular weight: A, B. B. Analysis of the capacity of the Light-inducible Bispecific T cell engager to trigger tumor cell killing in vitro: CTLs were dropped on a B16F10 cells monolayer in presence of the OptoFab protein and Mt-PhyB complex, and illuminated or not with a 656 nm light during 18 hours in the Optoplate at 37° C. Then, the CTL/target cells ratio, identified respectively with an anti-CD45 and an anti-TRP1 Ab, were analyzed by flow cytometry (representative of 2 independent experiments).

### EXAMPLE

### Material and Methods

### Cells Culture

Melanoma B16F10. The melanoma B16F10 cell line was cultured in the RPMI medium supplemented with 10% Fetal Bovine serum (FBS)-+ in humidified incubator of 92.5% air and 7.5% CO2 at 37° C. (ref innate??)

MCD4. These cells are 3A9m sub-line derived from mouse 3A9 CD4+ T cell hybridoma with high TCR expression as described (ref art yannick hamon scientif reports (2018)). The 3A9 CD4+ T cell hybridoma expresses on their surface a TCR specific for hen egg lysozyme peptide (HEL) bound to MHC II I-Ak molecules. Cells were cultured in cell culture medium (RPMI supplemented with 5% FBS, 1 mM NaPy, 10 mM Hepes) in humidified incubator of 90% air and 10% CO2 at 37° C.

HEK293T (human embryonic kidney 293T cell line) were cultured in cell culture medium (DMEM supplemented with 10% FBS, 1 mM NaPy, 2 mM L-Glu and geneticin) in humidified incubator of 92.5% air and 7.5% CO2.

Primary T Lymphocytes. CD8+ T cells were isolated from lymph nodes of C57BL/6 Rag1−/+OT-1−/+ mice and purified using the EasySep™ Mouse CD8+ T Cell Isolation Kit (STEMCELL Technologies) by negative selection. T cells were cultured in cell culture medium (DMEM/F-12 supplemented with 1 mM NaPy, 1% Nutridoma™-SP (Roche), 50U/ml Pen Strep, 10 mM Hepes, 0.05 mM b2-mercapto-ethanol).

Effector CD8 T cell. 6-well plates were coated with 3 ug/ml anti-CD38 (145-2C11) in PBS 4 h at 37° C. and washed three time with PBS prior to plating cells. CD8+ T cells were plated at 0.625.106 cells/ml in complete DMEM/F-F12 medium (DMEM/F-12 supplemented with 10% FBS, 1 mM NaPy, 10 mM Hepes, 50U/ml pen strep, 0.05 mM b2-mercapto-ethanol) with 1 μg/ml anti-CD28 (clone H37.51). The cells were cultured for 48 h (37° C., 5% CO2), after which IL-2 (PeproTech) was added to final concentrations of 10 U/ml. The cells were then cultured for a further 48 h.

### Generation and Production of the H57 OptoFab.

The H57 OptoFab was produced by cotransfecting HEK293T cells with pYD7-H57Fab-HC-PIF plasmid and pTT22-H57Fab-LC plasmid (ratio 1:3) using polyethylenimine (PEI, Polysciences). Cells were then maintained in production medium (DMEM supplemented with 2% FBS, 0.5% Tryptone TN1 (OrganoTechnie), 1.25 mM valproic acid and geneticin) at 37° C. with 5% CO2 in a humidified incubator. Supernatants were collected 7 days later and the H57 OptoFab were purified by Ni-NTA affinity chromatography. The purified protein buffer was exchanged to PBS using Slide-A-Lyzer™ Dialysis Cassette (Thermo Fischer).

H57 OptoFab
SEQ ID NO: 4 H57 Heavy Chain-PIF:
MEFGLSWVFLVALFRGVQCEVYLVESGGDLVQPGSSLKVSCAASG
FTFSDFWMYWVRQAPGKGLEWVGRIKNIPNNYATEYADSVRGRFT
ISRDDSRNSIYLQMNRLRVDDTAIYYCTRAGRFDHFDYWGQGTMV
TVSSASTKGPSVFPLAPSSKSTSGGTAALGCLVKDYFPEPVTVSW
NSGALTSGVHTFPAVLQSSGLYSLSSVVTVPSSSLGTQTYICNVN
HKPSNTKVDKRVEPKSCDKTGAGSGSGSGSGSMMFLPTDYCCRLS
DQEYMELVFENGQILAKGQRSNVSLHNQRTKSIMDLYEAEYNEDF
MKSIIHGGGGAITNLGDTQVVPQSHVAAAHETNMLESNKHVDGSG
SGSGSGSENLYFQGHHHHHH*
SEQ ID NO:5 H57 Light Chain:
MKYLLPTAAAGLLLLAAQPAMAYELIQPSSASVTVGETVKITCSG
DQLPKNFAYWFQQKSDKNILLLIYMDNKRPSGIPERFSGSTSGTT
ATLTISGAQPEDEAAYYCLSSYGDNNDLVFGSGTQLTVLRGRTVA
APSVFIFPPSDEQLKSGTASVVCLLNNFYPREAKVQWKVDNALQS
GNSQESVTEQDSKDSTYSLSSTLTLSKADYEKHKVYACEVTHQGL
SSPVTKSENRGEC*
SEQ ID NO: 2 HoloPhyB:
MVSGVGGSGGGRGGGRGGEEEPSSSHTPNNRRGGEQAQSSGTKSL
RPRSNTESMSKAIQQYTVDARLHAVFEQSGESGKSFDYSQSLKTT
TYGSSVPEQQITAYLSRIQRGGYIQPFGCMIAVDESSFRIIGYSE
NAREMLGIMPQSVPTLEKPEILAMGTDVRSLFTSSSSILLERAFV
AREITLLNPVWIHSKNTGKPFYAILHRIDVGVVIDLEPARTEDPA
LSIAGAVQSQKLAVRAISQLQALPGGDIKLLCDTVVESVRDLTGY
DRVMVYKFHEDEHGEVVAESKRDDLEPYIGLHYPATDIPQASRFL
FKQNRVRMIVDCNATPVLVVQDDRLTQSMCLVGSTLRAPHGCHSQ
YMANMGSIASLAMAVIINGNEDDGSNVASGRSSMRLWGLVVCHHT
SSRCIPFPLRYACEFLMQAFGLQLNMELQLALQMSEKRVLRTQTL
LCDMLLRDSPAGIVTQSPSIMDLVKCDGAAFLYHGKYYPLGVAPS
EVQIKDVVEWLLANHADSTGLSTDSLGDAGYPGAAALGDAVCGMA
VAYITKRDFLFWFRSHTAKEIKWGGAKHHPEDKDDGQRMHPRSSF
QAFLEVVKSRSQPWETAEMDAIHSLQLILRDSFKESEAAMNSKVV
DGVVQPCRDMAGEQGIDELGAGTLEKLVDGAGSWSHPQFEKENLY
FQGLEHHHHHH*

### Measurement of Light-Induced Tumor Cell Killing by Flow Cytometry

Melanoma B16F10 cells were used as targeted tumor cell model. A total of 0.6.1056 target cells per well were plated in a 96-well plate and loaded with the Mt-PhyB complex. Effector CD8 T cells were cocultured at 10:1 ratio with the target cells in a total volume of 200 μl of RPMI supplemented with 10% FBS, 1 mM NaPy, 10 mM Hepes, 0.05 mM b2-mercapto-ethanol per well. The H57 OptoFab protein (0.324 μg/mL) and the Mt-PhyB complex were added in solution. Cells were illuminated with the indicated light for 18 hours at 37° C. Then, the CTL/target cells ratio, identified respectively with an anti-CD45 and an anti-TRP1 Ab, were determined by flow cytometry.

Spatial Targeting of Tumor Cell Killing with Light In Vitro.

A total of 1.106 target cells were plated in Lab Tek chamber slides and loaded with the Mt-PhyB complex. Effector CD8 T cells were loaded with the PBX calcium reporter and with the H57 OptoFab, then dropped on the B16F10 monolayer, under a videomicroscope at 37° C. An area at the center of field has been enlighten with a 656 nm light for 2 hours. Then, CTLs were washed out and the apoptotic B16F10 tumoral cells, labelled with the CellEvent caspase-3/7 Green detection reagent (Invitrogen).

### Results:

### Design of the OptoFab (Also Called LiTe): A Recombinant Light-Inducible TCR Agonist for Untouched Primary Murine T Cells:

Our first goal was to develop a versatile and non-invasive light-responsive stimulatory module to control untouched primary T cell activation. We thus developed a new class of optogenetics-based recombinant molecules able to reversibly trigger TCR signaling in response to specific wavelength of light. These molecules are composed of a Fab fragment derived from an agonistic antibody targeting the TCR, linked to an optogenetic domain that allows its light induced oligomerization/immobilization. The rational is that a monovalent Fab fragment derived from an agonistic antibody often keeps the specificity for the ligand, but loses the agonistic property. However, when immobilized or oligomerized, it recovers its agonistic capacity.

The H57-597 monoclonal antibody, an agonistic antibody specific for the C domain of the TCRβ chain, and its derived monovalent Fab fragment showed such characteristics. Indeed, the soluble H57-Fab fragment loaded on naïve T cells did not induce any TCR signaling. However, once immobilized on a coverslip, it triggered a potent TCR signaling visualized by the measurement of the intracellular calcium influxes in T cell lines constitutively expressing the calcium sensor Twitch2B (Thestrup T, Griesbeck O. Nat Methods. 2014) (data not shown). Therefore, we generated a recombinant protein composed of the H57-Fab fragment coupled to the Phytochrome Interacting Factor 6 (PIF6), a domain belonging to the PIF/PhyB (Phytochrome B of A. Thaliana) optogenetic pair. We named this molecule the H57 OptoFab (data not shown). The Phytochrome B of Arabidopsis thaliana, when exposed to a light at 650 nm, open a binding site for PIF6. An exposure to a light at 730 nm reverses this process [6,7] (data not shown). The principle of the molecular system we developed is to use PhyB coated on beads or on any surface to reversibly capture TCR bound H57-OptoFab, and thus control TCR stimulations, by applying light of specific wavelength (data not shown).

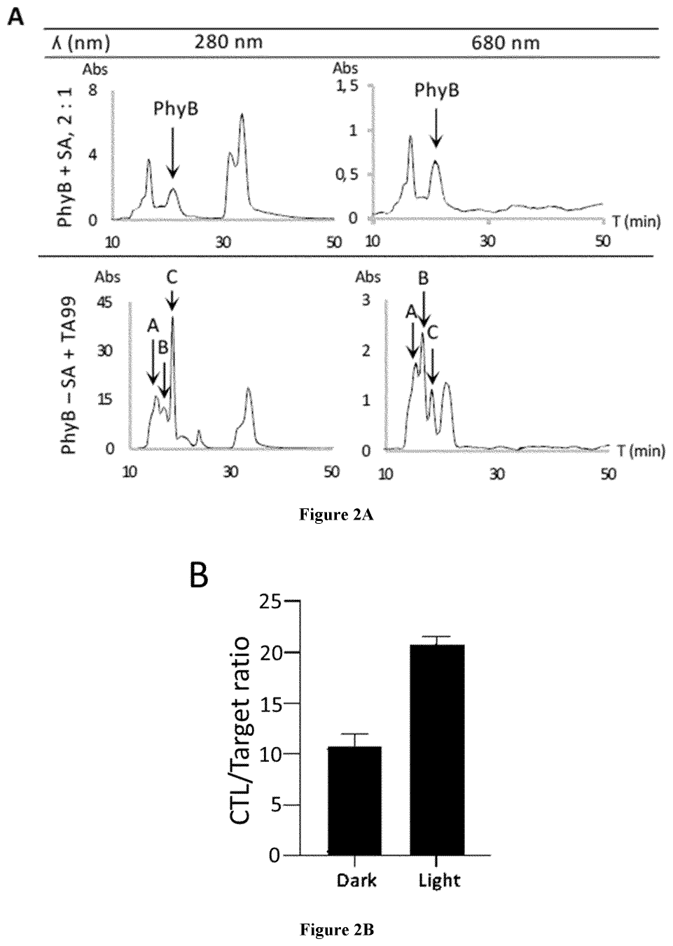

PIF6 domain was cloned at the C-terminus of the Heavy Chain part of the H57-597 derived Fab. Then, the H57-OptoFAb was produced in HEK cells, purified by affinity chromatography, and its binding capacity to the TCR was evaluated by flow cytometry (FIG. 2A). The labelling of mCD4 hybridoma with the H57-OptoFab revealed that it bound to TCR expressing cells. To verify that the PIF6 peptide didn't alter the Fab specificity to the TCR C□ epitope, a competition assay between the H57 OptoFab and a conventional AF488-H57 Fab was performed (data not shown). Whereas the AF488-H57 Fab labelled mCD4 T cells, this labelling was abrogated when the H57-AF488 Ab is co-incubated with an excess of H57-OptoF Ab (data not shown). These experiments showed that the H57-OptoFab is specific for the TCR β chain and that its coupling to the PIF6 domain didn't alter its specificity.

In parallel, the N-terminal PhyB 1-651 region, the minimal region of PhyB able to reversibly respond to light exposure, was produced in E. coli and affinity purified (HoloPhyB, Leung D, Rosen M, PNAS 2008). Then, to have a read on HoloPhyB functionality, we first test its photoswitching capacity by spectrophotometry (data not shown). As expected, an exposure of HoloPhyB to a light at 656 nm modified its absorption spectrum and switched it from its pR to its pFR form. HoloPhyB came back to its pR form following an exposure to a light at 730 nm. Finally, we performed a pull-down experiment to verified that the H57 OptoFab was efficiently captured on HoloPhyB-coated beads in response to light. The western blot analysis of this assay clearly showed that the H57 OptoFab was bound to HoloPhyB upon 656 nm light exposure and was released upon 730 nm light exposure or in darkness (data not shown). Therefore, we designed and produced a recombinant H57-derived OptoFab targeting the TCRβ chain that can be captured and released by HoloPhyB in response to specific wavelength of light.

### OptoFab Provides an Accurate Control of TCR Stimulation

We then evaluated the capacity of the H57-OptoFab module to provide reversible light-controlled TCR stimulations to untouched primary T cells. In T cells, intracellular calcium fluxes are rapidly triggered following TCR stimulations, and stop few seconds after signal termination (dustin). Hence, intracellular calcium measurements constitute an accurate read-out of the dynamics of TCR stimulations. Therefore, primary CD8 T cells were extracted from mice lymph nodes, loaded with PBX calcium-sensitive dye and the H57 OptoFab, then incubated with HoloPhyB-coated beads and imaged with a videomicroscope at 37° C. Whereas under 730 nm illumination, the intracellular calcium levels in primary T cells were low, an 656 nm illumination triggered a rapid calcium elevation in cells contacting HoloPhyB-coated beads (data not shown). Fluorescence intensity quantifications revealed that T cell responded in a synchronized manner at the population scale (data not shown). Within 30 s after 656 nm light exposure, most of the T lymphocytes in contact with beads showed a strong increased in intracellular calcium concentration, which returned back to a level similar to the resting state in less than 2 minutes following 730 nm illumination. Calcium influx constitute a very sensitive read-out of TCR stimulation as one or few engaged TCR are sufficient to induce a significant calcium increase (Trautman, davis). Thus, the decrease of the cell fluorescence to the resting state level following the 730 nm illumination suggested that all the TCR were freed under inhibitory conditions. It underlined the remarkable reversibility of the system. Furthermore, we observed that the OptoFab system permitted to deliver iterative stimulation/resting cycles to the T cells, translated by calcium influxes at each 656 nm light photostimulation that stopped when the light wavelength is switched at 730 nm (data not shown) Also, analyses at higher magnification of these time-lapse movies revealed that at each 656 nm light photostimulation, the shape of the T lymphocytes changed to match the shape of the beads, concomitantly to the calcium elevation (data not shown). This shape is reminiscent of T cell shape reported in the early immunological synapse and suggest that a large number of TCR are captured by the beads in response to photostimulations.

Altogether, these experiments showed that the OptoFab system provide for the first time a light-response capacity to untouched primary T cell. This cellular ON/OFF switch permitted the accurate spatiotemporal control of the T cell activation by delivering strong and reversible TCR stimulations.

### OptoFab-Driven TCR Photostimulation Leads to Full T Cell Activation

As calcium constitute a proximal signaling event downstream TCR, we next evaluated the capacity of the OptoFab/PhyB system to fully activate untouched primary CD8 T cells. Purified CD8 T cells were incubated with the OptoFab and PhyB-coated beads, in presence or not of a CD28 stimulating antibody. They were placed on an Optoplate and illuminated 18 hours with light pulses at 630 nm (stimulation), or at 780 nm (inhibition). Then, their activation statue has been evaluated by analyzing the level of expression of the molecules CD69, CD62-L or CD25 at the T cell surface by flow cytometry. Upon 780 nm light, the cells stayed in resting state. In contrast, the 630 nm light triggered a potent T cell activation visualized by the increase of the expression of CD69 and CD25 at the T cell surface, and the decrease of CD62-L (data not shown). As expected, the 630 nm light in absence of the full OptoFab system alone didn't drive T cell activation. Importantly, the low light power required to trigger efficient T cell stimulation or to keep cells in an inactivated state, 0.14 mW·cm⁻² at 630 nm and 2.8 mW·cm⁻² at 780 nm respectively, didn't induce any detectable phototoxicity on an 18h illumination period (data not shown).

To have a more functional read of the T cell activation, we analyzed the IL-2 production in the supernatant the differentially stimulated T cells (data not shown). These experiments showed that the H57 OptoFab stimulation system efficiently triggers IL2 production, only under a 630 nm light exposure. In these conditions, its efficacy was similar to the one of a coated anti-CD3 antibody.

Therefore, the OptoFab system provided the capacity to trigger the full activation of untouched primary T cells with light, leading to the set-up of their effector functions such as cytokines secretion.

### Design of a Derivative of the OptoFab System Optimized for the Photocontrol of the Tumor Cell Killing by Murine T Cells

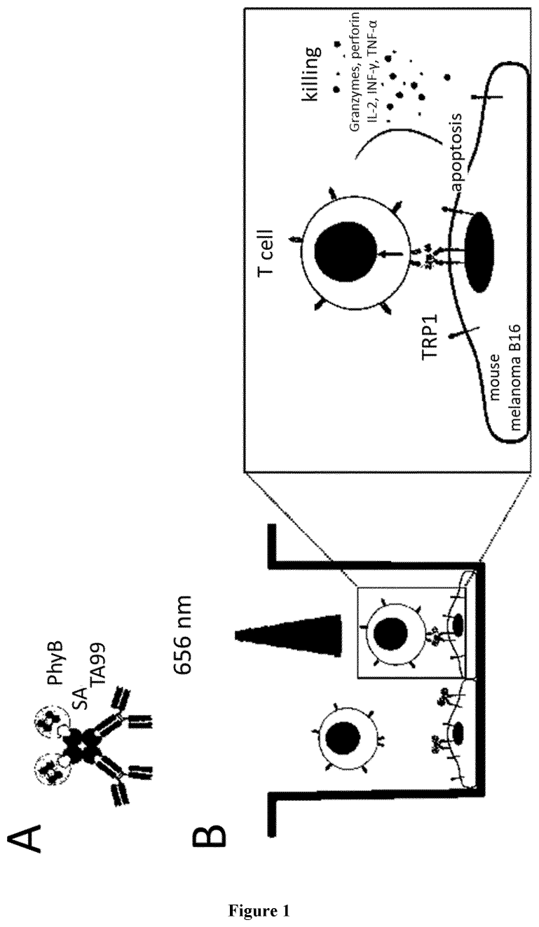

The OptoFab/PhyB system induces T cells activation by immobilizing or aggregating the TCR on a PhyB-coated surface. When PhyB is coated on beads, we noticed that T cells generated membrane extensions toward the beads and formed a junction that is reminiscent of the immune synapse. We thus decided to create a new version of the OptoFab/PhyB system that allowed to target PhyB on tumor cells to control in space and time tumor cell lysis by CTLs, with light (FIG. 1A-B). We named this molecular system composed of the Melanoma-targeted PhyB and the LiTe, LiTe-Me for LiTe targeting Melanoma. Therefore, we have biotinylated an antibody targeting TRP-1 (the TA-99 antibody), a molecule expressed at the surface of melanoma cells, and generated molecular complex composed of streptavidin, biotinylated TA-99 and biotinylated PhyB in a 1/2/2 ratio. We named this molecular complex, designed to address PhyB at surface of melanoma cells, the Mt-PhyB for Melanoma-targeted PhyB. Mt-PhyB complexes were purified by HPLC, as high molecular weight complexes formed upon TA-99 addition (peak A and B, FIG. 2A). The presence of PhyB was verified by measuring the absorption of the complexes at 680 nm (FIG. 2A, right panel), and confirmed by a western blot analysis (data not shown). In addition, the capacity of Mt-PhyB complexes to bind to melanoma cells have been confirmed by flow cytometry (data not shown).

We then evaluated the capacity of the OptoFab/Mt-PhyB (LiTe-Me) module to trigger the tumor cell killing by CTL in response to photostimulations. B16 melanoma cells were coculture with CTLs in presence of the OptoFab/Mt-PhyB module, and enlighten overnight or not with the stimulatory light. Then, the CTL/B16 cell ratios were measured by flow cytometry (FIG. 2C). These experiments showed a two-fold increase of CTL/B16 ratio when the cells are exposed to the stimulatory light in presence of the OptoFab/Mt-PhyB (LiTe-Me) module. This effect is specific to the activated OptoFab/Mt-PhyB module, as no changes in CTL/B16 ratio were observed when cells were exposed to 630 nm light in absence of the complex (data not shown). Therefore, these experiments showed that OptoFab/Mt-PhyB module triggers the killing of B16 melanoma cells by murine CTLs in response to light.

Light represents a very accurate stimulus as it could be controlled finely in time but also in space. We thus decided to verify if OptoFab/Mt-PhyB (LiTe-Me) module allows a precise spatial control of CTL-driven B16 killing. To do so, B16 and CTLs were placed in a microscopy chamber in presence of the OptoFab/Mt-PhyB (LiTe-Me) module. A light of a 656 nm wavelength has been focused in a region at the center of the microscope field during 2 hours, then the T cells were removed by washing, and the apoptotic tumor cells detected using a specific caspase 3-7 activity sensor. After 2 hours of illumination, we observed clusters of cells with an increased caspase 3-7 activity specifically in the area illuminated with the 656 nm light (data not shown). It suggested that the Lite-Me (OptoFAb/Mt-PhyB complex) induced the melanoma cells killing by the T cells in response to light. Altogether, these results showed that theLite-Me (OptoFab/Mt-PhyB module) allow the spatio-temporal control of the tumor cell killing by CTLs in vitro, in response to light.

### REFERENCES

Throughout this application, various references describe the state of the art to which this invention pertains. The disclosures of these references are hereby incorporated by reference into the present disclosure.
1. Shi, H., Sadler, P. J. How promising is phototherapy for cancer?. Br J Cancer 123, 871-873 (2020).
2. Abrahamse H, Hamblin MR. New photosensitizers for photodynamic therapy. Biochem J. 2016; 473 (4): 347-364.
3. Vigneron N, Stroobant V, Van den Eynde BJ, van der Bruggen P. Database of T cell-defined human tumor antigens: the 2013 update. Cancer Immun. 2013 Jul. 15; 13:15.

1. A light-controlled molecular system comprising:
a. at least one recombinant protein comprising a variable domain of an antibody that is fused at its c-terminal end to a compound that interacts with a photoactivable agent in a light-dependent manner, and
b. at least one tumor-antigen targeting antibody that is fused at its c-terminal end to the photoactivable agent.

2. The light-controlled molecular system of claim 1, wherein the at least one recombinant protein comprises a Fab fragment wherein the VH domain of the Fab fragment is fused at its c-terminal end to the compound that can interact with a photoactivable agent in a light-dependent manner.

3. The light-controlled molecular system of claim 2, wherein the Fab fragment is from an agonistic antibody specific for a receptor of an immune cell.

4. The light-controlled molecular system of claim 1, wherein the recombinant protein of the present invention comprises a single domain antibody, and in particular or an agonistic single domain antibody.

5. The light-controlled molecular system of claim 3, wherein the agonistic antibody is specific for a TCR or a costimulatory receptor selected from the group consisting of CD134 (OX40), CD137 (4-1BB), CD28, GITR, CD27, CD70, ICOS, RANKL, TNFRSF25 (DR3), CD258 (LIGHT), CD40 and HVEM.

6. The light-controlled molecular system of claim 3, wherein the agonistic antibody is specific for TCR Beta or CD3epsilon

7. The light-controlled molecular system of claim 1, wherein the at least one tumor-antigen targeting antibody is a single-domain antibody or an antibody mimetics.

8. The light-controlled molecular system of claim 1, wherein the compound that interacts with a photoactivable agent in a light-dependent manner is a factor that interacts with a photoreceptor protein in a light-dependent manner and the photoactivable agent fused to the at least one tumor-antigen targeting antibody is the photoreceptor protein.

9. The light-controlled molecular system of claim 8, wherein the factor is selected from the group consisting of PIF1, PIF2, PIF3, PIF4, PIF5, PIF6, and PIF7,

10. The light-controlled molecular system of claim 9, wherein the factor comprises an amino acid sequence that has at least 90% of identity with the amino acid sequence as set forth in SEQ ID NO:1.

11. The light-controlled molecular system according to claim 8, wherein the photoreceptor protein is selected from the group consisting of Phytochrome A (PhyA), Phytochrome B (PhyB), Phytochrome C (PhyC), Phytochrome D (PhyD), and Phytochrome E (PhyE).

12. The light-controlled molecular system according to claim 11, wherein the photoreceptor protein comprises an amino acid sequence that has at least 90% identity with the amino acid sequence as set forth in SEQ ID NO:2 or SEQ ID NO: 3.

13. The light-controlled molecular system according to claim 1, wherein the compound that interacts with a photoactivable agent in a light-dependent manner is the photoactivable agent fused to the at least one tumor-antigen targeting antibody, wherein the photoactivable agent dimerizes in a light-dependent manner.

14. The light-controlled molecular system according to claim 13, wherein the photoactivable agent is a photoreceptor protein or a photoisomerizable compound.

15. A method of activating on demand an immune cell or a plurality of immune cells comprising:
i. contacting the immune cell or the plurality of immune cells with the light-controlled molecular system according to claim 1, and
ii. exposing the cell or the plurality of immune cells to a suitable wavelength of light wherein said exposing causes oligomerization of a complex binding the at least one recombinant protein of said light-controlled molecular system to the at least one tumor-antigen targeting antibody of said light-controlled molecular system.

16. The method according to claim 15, wherein the plurality of immune cells is embedded in a tissue, organ or organism.

17. The method according to claim 15, wherein the immune cells are lymphocytes; natural killer cells; or myeloid cells.

18. A method for treating cancer comprising a tumor in a subject in need thereof, comprising:
i. administering to said subject the light-controlled molecular system according to claim 1; and
ii. exposing the tumor to a suitable wavelength of light wherein said exposing causes formation of a complex binding the at least one recombinant protein to the at least one tumor antigen targeting antibody.

19. The method according to claim 18, wherein the cancer is selected in the group consisting n of: head and neck squamous cell carcinoma (HNSCC); adrenal cortical cancer; anal cancer; periphilar cancer; distal bile duct cancer; intrahepatic bile duct cancer; osteoblastoma; osteochrondroma; hemangioma; chondromyxoid fibroma; astrocytoma; ductal carcinoma in situ; gynecomastia; endometrial adenocarcinoma; adenocanthoma; papillary serous adenocarcinoma; laryngeal and hypopharyngeal cancer; hemangioma, hepatic adenoma; focal nodular hyperplasia; small cell lung cancer; non-small cell lung cancer; mesothelioma, plasmacytoma; esthesioneuroblastoma; midline granuloma; nasopharyngeal cancer; oral cavity and oropharyngeal cancer, ovarian cancer; pancreatic cancer; penile cancer; pituitary cancer; prostate cancer; salivary gland cancer; non-melanoma skin cancer; stomach cancer, testicular cancer; thymus cancer; follicular carcinoma; anaplastic carcinoma; poorly differentiated carcinoma; medullary thyroid carcinoma; vaginal cancer, vulvar cancer, uterine leiomyosarcoma; bladderneoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating ductal carcinoma; medullary carcinoma; infiltrating lobular carcinoma; lobular carcinoma in situ; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; leydig cell tumor, malignant; lipid cell tumor, malignant; paraganglioma, malignant; gliomas; medulloblastoma; Schwannoma; germinoma; craniopharyngioma; extra-mammary paraganglioma, malignant; phcochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; chorioadenoma destruens; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

20. The light-controlled molecular system of claim 7 wherein the single-domain antibody is an anti-TRP-1 single domain antibody or an anti-EpCAM single domain antibody.
